(19) United States
(12) Patent Application Publication
(10) Pub. No.: US 2008/0026943 A1
(43) Pub. Date: Jan. 31, 2008

(54) Phenyl-Substituted [1,2]-Oxazine-3,5-Dione and Dihydropyrone Derivatives

(76) Inventors: Reiner Fischer, Monheim (DE); Thomas Bretschneider, Lohmar (DE); Folker Lieb, Leverkusen (DE); Thomas Schenke, Bergisch Gladbach (DE); Astrid Ullmann-Koppold, Leverkusen (DE); Mark Wilhelm Drewes, Langenfeld (DE); Dieter Feucht, Eschborn (DE); Jorg Konze, Koln (DE); Peter Losel, Leverkusen (DE); Olga Malsam, Rosrath (DE); Karl-Heinz Kuck, Langenfeld (DE); Ulrike Wachendorff-Neumann, Neuwied (DE); Alfred Angermann, Kriftel (DE); Christian Arnold, Langenfeld (DE); Thomas Auler, Leichlingen (DE); Waltraud Hempel, Liederbach (DE); Martin Jeffrey Hills, Idstein (DE); Heinz Kehne, Hofheim (DE); Erich Sanwald, Kiel (DE)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/631,656

(22) PCT Filed: Jun. 22, 2005
(86) PCT No.: PCT/EP05/06740

§ 371 Date: Sep. 19, 2007

(30) Foreign Application Priority Data
Jul. 5, 2004 (DE) 10 2004 032 421.2

(51) Int. Cl. A61K 31/535; A01N 43/02; A01N 43/40; A01N 43/72; A01P 1/00; C07D 315/00; C07D 265/02; A01P 13/00; A01P 21/00; A61K 31/351

(52) U.S. Cl. 504/130; 504/140; 504/223; 504/291; 514/228.8; 514/460; 544/63; 549/417

(57) ABSTRACT

The present invention relates to new phenyl-substituted [1,2]-oxazine-3,5-dione derivatives and dihydropyrone derivatives of the formula (I) 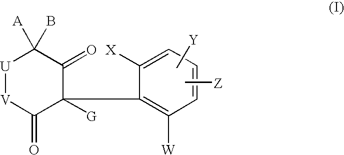 in which A, B, U, V, W, X, Y and Z have the definition indicated above, to a number of processes for preparing them and to their use as microbicides and/or pesticides and/or herbicides.

The present invention relates to new phenyl-substituted [1,2]-oxazine-3,5-dione derivatives and dihydropyrone derivatives, to a number of processes for preparing them and to their use as microbicides and/or pesticides and/or herbicides.

The invention also relates to new selective-herbicidal active-compound combinations which comprise firstly phenyl-substituted [1,2]-oxazine-3,5-dione derivatives or phenyl-substituted dihydropyrone derivatives and secondly at least one crop plant tolerance promoter compound and can be used with particular success for selective weed control in various crops of useful plants.

Not only 4-halo-4-phenyl- and 4-nitro-4-phenyl-substituted [1,2]-oxazine-3,5-dione derivatives but also 3-halo-3-phenyl- and 3-nitro-3-phenyl-substituted dihydropyrone derivatives are unknown to date in the literature. Only 3-halo-3-phenyl-quinoline-2,4-dione derivatives have been described, for example by Kafku, S. et al. in Heterocycles 57, 1659-1682, (2002).

Additionally, 3-nitro-3-phenyl-quinoline-2,4-dione derivatives are known, for example from Stadlbauer, W. et al. from J. Heterocyclic Chemistry 29, 1535-1540, (1992).

A crop protection application for these compounds has not been described.

New compounds of the formula (I) have now been found 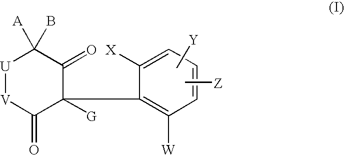
in which
U represents oxygen or represents a group 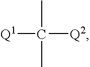
V represents oxygen or represents a group 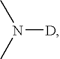
with the proviso that U and V do not simultaneously represent oxygen, although one of the substituents must represent oxygen,
W represents hydrogen, halogen, alkyl, alkenyl, alkynyl, alkoxy, alkenyloxy, haloalkyl or haloalkoxy,
X represents halogen, alkyl, alkenyl, alkynyl, alkoxy, alkenyloxy, alkylthio, alkylsulphinyl, alkylsulphonyl, haloalkyl, haloalkoxy, haloalkenyloxy, nitro or cyano,
Y represents hydrogen, halogen, alkyl, alkoxy, alkenyloxy, haloalkyl, haloalkoxy, haloalkenyloxy, nitro or cyano,
Z represents hydrogen, alkyl, alkenyl, alkynyl, alkoxy, halogen or in each case optionally substituted aryl or hetaryl,
A represents hydrogen, in each case optionally halogen-substituted alkyl, alkenyl, alkoxyalkyl, polyalkoxyalkyl, alkylthioalkyl, saturated or unsaturated, optionally substituted cycloalkyl, in which optionally at least one ring atom has been replaced by a heteroatom,
B represent hydrogen, alkyl or alkoxyalkyl, or
A and B, together with the carbon atom to which they are attached, represent a saturated or unsaturated, unsubstituted or substituted ring optionally containing at least one heteroatom,
D represents hydrogen or an optionally substituted radical from the group consisting of alkyl, alkenyl, alkoxyalkyl, polyalkoxyalkyl, alkylthioalkyl and saturated or unsaturated cycloalkyl in which optionally one or more ring members have been replaced by heteroatoms,
G represents halogen or nitro,
A and Q¹ together represent optionally substituted alkanediyl in which two carbon atoms that are not directly adjacent optionally form a further optionally substituted ring which may optionally be interrupted with a heteroatom,
Q¹ represents hydrogen, alkyl, alkoxy, alkoxyalkyl, optionally substituted cycloalkyl (in which optionally a methylene group has been replaced by oxygen or sulphur) or in each case optionally substituted phenyl, hetaryl, phenylalkyl or hetarylalkyl,
Q² represents hydrogen or alkyl,
Q¹ and Q², together with the carbon atom to which they are attached, represent a saturated or unsaturated, unsubstituted or substituted ring optionally containing a heteroatom.

The compounds of the formula (I), as they are and as a function of the nature of the substituents, may be present in the form of geometric and/or optical isomers or isomer mixtures, in different compositions, which optionally can be conventionally separated. Not only the pure isomers but also the isomer mixtures, their preparation and use, and also compositions comprising them, are provided by the present invention. Below, however, for the sake of simplicity, explicit reference is always to compounds of the formula (I), although this means not only the pure compounds but also, where appropriate, mixtures with different fractions of isomeric compounds.

The compounds of the formula (I) may be present both as mixtures and in the form of their pure isomers. Mixtures of compounds of the formula (I) can be separated optionally in conventional manner by physical methods, such as by chromatographic methods, for example.

Below, in order not to complicate things unnecessarily, in each case only one of the possible isomers is shown. It is not intended to rule out the possibility of the compounds, where appropriate, being in the form of the isomer mixtures or in the respective other isomeric form.

Including U and V for the groups 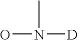
and 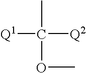
results in the following primary structures (I-1) and (I-2) 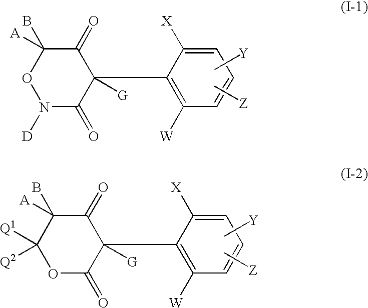
in which
A, B, D, G, Q¹, Q², W, X, Y and Z have the definitions indicated above.
A) Additionally it has been found that compounds of the formulae (I-1) to (I-2) 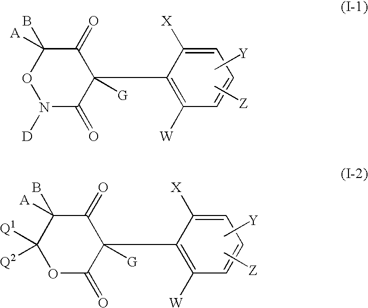
   in which
   A, B, D, G, Q¹, Q², W, X, Y and Z have the definitions indicated above
   and
   G represents halogen, preferably chlorine or bromine,
   are obtained if compounds of the formulae (II-1) and (II-2), 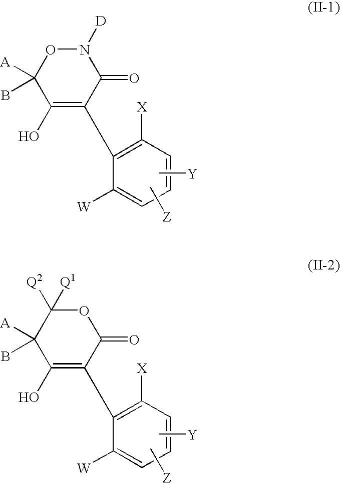
   in which
   A, B, D, Q¹, Q², W, X, Y and Z have the definition indicated above,
   are reacted with halogenating agents in the presence of a solvent and optionally in the presence of a free-radical initiator.
B) Moreover, compounds of the formulae (I-1) to (I-2) 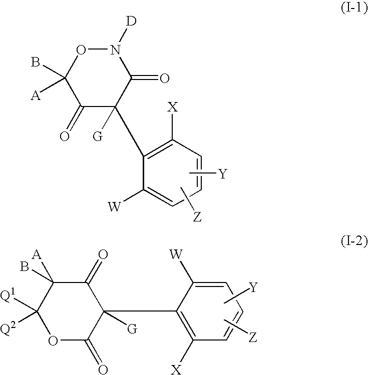
   in which
   A, B, D, Q¹, Q², W, X, Y and Z have the definitions indicated above
   and
   G represents nitro
   are obtained if compounds of the formulae (II-1) and (II-2), 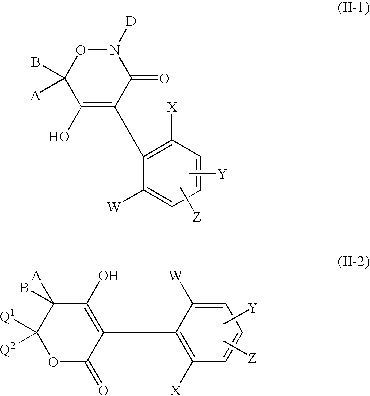
   in which
   A, B, D, Q¹, Q², W, X, Y and Z have the definition indicated above,
   are reacted with nitrating reagents such as fuming nitric acid, for example, in the presence of a solvent.

Some of the compounds required for processes A and B, of the formulae (II-1) to (II-2) 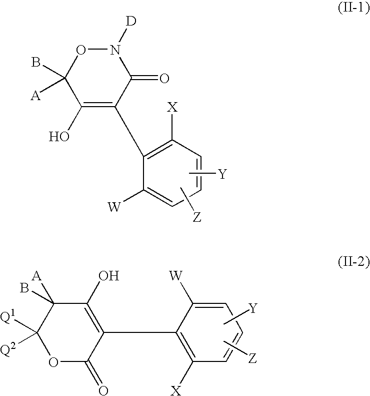
in which
A, B, D, Q¹, Q², W, X, Y and Z have the definition indicated above,
are known: EP-A-394889, WO 92/07837, U.S. Pat. No. 5,728,831, WO 01/17972, WO 01/98288, WO 03/048138 or these compounds can be synthesized by the processes specified in principle therein.

Suitable halogenating agents for process A include, for example, sulphuryl chloride, sulphuryl bromide, thionyl chloride, thionyl bromide, imides, such as N-bromosuccinimide or N-chlorosuccinimide, chlorosulphonic acid, and also hypochlorites, such as tert-butyl hypochlorite, for example.

Suitable nitrating reagents for process B include fuming nitric acid, and also “nitrating acid mixtures”.

Additionally it has been found that the new compounds of the formula (I) exhibit very good activity as pesticides, preferably as insecticides and/or acaricides and/or microbicides and/or as herbicides.

Surprisingly it has now also been found that certain substituted, cyclic keto enols, when employed together with the crop plant tolerance promoter compounds (safeners/antidotes) described later on, are extremely good at preventing damage to the crop plants and can be used with particular advantage as broad-spectrum combination products for the selective control of unwanted plants in crops of useful plants, such as in cereals but also in maize, soya and rice, for example.

The invention also provides selective-herbicidal compositions comprising an effective amount of an active-compound combination comprising as components
(a′) at least one phenyl-substituted [1,2]-oxazinedione derivative of the formula (I-1) or one phenyl-substituted dihydropyrone derivative of the formula (I-2), in which A, B, D, G, Q¹, Q², W, X, Y and Z have the definition indicated above
and
(b′) at least one crop plant tolerance promoter compound from the following group of compounds:
   4-dichloroacetyl-1-oxa-4-azaspiro[4.5]decane (AD-67, MON-4660), 1-dichloro-acetylhexahydro-3,3,8a-trimethylpyrrolo[1,2-a]pyrimidin-6(2H)-one (dicyclonon, BAS-145138), 4-dichloroacetyl-3,4-dihydro-3-methyl-2H-1,4-benzoxazine (benoxacor), 1-methylhexyl 5-chloroquinoline-8-oxyacetate (cloquintocet-mexyl—cf. also related compounds in EP-A-86750, EP-A-94349, EP-A-191736, EP-A-492366), 3-(2-chlorobenzyl)-1-(1-methyl-1-phenylethyl)urea (cumyluron), α-(cyanomethoximino)phenylacetonitrile (cyometrinil), 2,4-dichlorophenoxyacetic acid (2,4-D), 4-(2,4-dichlorophenoxy)butyric acid (2,4-DB), 1-(1-methyl-1-phenylethyl)-3-(4-methylphenyl)urea (daimuron, dymron), 3,6-dichloro-2-methoxybenzoic acid (dicamba), S-1-methyl-1-phenylethyl piperidine-1-thiocarboxylate (dimepiperate), 2,2-dichloro-N-(2-oxo-2-(2-propenylamino)ethyl)-N-(2-propenyl)acetamide (DKA-24), 2,2-dichloro-N,N-di-2-propenylacetamide (dichlormid), 4,6-dichloro-2-phenylpyrimidine (fenclorim), ethyl 1-(2,4-dichlorophenyl)-5-trichloromethyl-1H-1,2,4-triazole-3-carboxylate (fenchlorazole-ethyl—cf. also related compounds in EP-A-174562 and EP-A-346620), phenylmethyl 2-chloro-4-trifluoromethylthiazole-5-carboxylate (flurazole), 4-chloro-N-(1,3-dioxolan-2-ylmethoxy)-α-trifluoroacetophenone oxime (fluxofenim), 3-dichloroacetyl-5-(2-furanyl)-2,2-dimethyloxazolidine (furilazole, MON-13900), ethyl 4,5-dihydro-5,5-diphenyl-3-isoxazolecarboxylate (isoxadifen-ethyl—cf. also related compounds in WO-A-95/07897), 1-(ethoxycarbonyl)ethyl 3,6-dichloro-2-methoxybenzoate (lactidichlor), (4-chloro-o-tolyloxy)acetic acid (MCPA), 2-(4-chloro-o-tolyloxy)propionic acid (mecoprop), diethyl 1-(2,4-dichorophenyl)-4,5-dihydro-5-methyl-1H-pyrazole-3,5-dicarboxylate (mefenpyr-diethyl—cf. also related compounds in WO-A-91/07874), 2-dichloromethyl-2-methyl-1,3-dioxolane (MG-191), 2-propenyl-1-oxa-4-azaspiro[4.5]decane-4-carbodithioate (MG-838), 1,8-naphthalic anhydride, α-(1,3-dioxolan-2-ylmethoximino)phenylacetonitrile (oxabetrinil), 2,2-dichloro-N-(1,3-dioxolan-2-ylmethyl)-N-(2-propenyl)acetamide (PPG-1292), 3-dichloro-acetyl-2,2-dimethyloxazolidine (R-28725), 3-dichloroacetyl-2,2,5-trimethyl-oxazolidine (R-29148), 4-(4-chloro-o-tolyl)butyric acid, 4-(4-chlorophenoxy)butyric acid, diphenylmethoxyacetic acid, methyl diphenylmethoxyacetate, ethyl diphenylmethoxyacetate, methyl 1-(2-chlorophenyl)-5-phenyl-1H-pyrazole-3-carboxylate, ethyl 1-(2,4-dichlorophenyl)-5-methyl-1H-pyrazole-3-carboxylate, ethyl 1-(2,4-dichlorophenyl)-5-isopropyl-1H-pyrazole-3-carboxylate, ethyl 1-(2,4-dichloro-phenyl)-5-(1,1-dimethylethyl)-1H-pyrazole-3-carboxylate, ethyl 1-(2,4-dichloro-phenyl)-5-phenyl-1H-pyrazole-3-carboxylate (cf. also related compounds in EP-A-269806 and EP-A-333131), ethyl 5-(2,4-dichlorobenzyl)-2-isoxazoline-3-carboxylate, ethyl 5-phenyl-2-isoxazoline-3-carboxylate, ethyl 5-(4-fluorophenyl)-5-phenyl-2-isoxazoline-3-carboxylate (cf. also related compounds in WO-A-91/08202), 1,3-dimethylbut-1-yl 5-chloroquinoline-8-oxyacetate, 4-allyloxybutyl 5-chloro-quinoline-8-oxyacetate, 1-allyloxyprop-2-yl 5-chloroquinoline-8-oxyacetate, methyl 5-chloroquinoxaline-8-oxyacetate, ethyl 5-chloroquinoline-8-oxyacetate, allyl 5-chloroquinoxaline-8-oxyacetate, 2-oxoprop-1-yl 5-chloroquinoline-8-oxyacetate, diethyl 5-chloroquinoline-8-oxymalonate, diallyl 5-chloroquinoxaline-8-oxymalonate, diethyl 5-chloroquinoline-8-oxymalonate (cf. also related compounds in EP-A-582198), 4-carboxychroman-4-ylacetic acid (AC-304415, cf. EP-A-613618), 4-chlorophenoxyacetic acid, 3,3′-dimethyl-4-methoxybenzophenone, 1-bromo-4-chloromethylsulphonylbenzene, 1-[4-(N-2-methoxybenzoylsulphamoyl)-phenyl]-3-methylurea (also known as N-(2-methoxybenzoyl)-4-[(methylamino-carbonyl)amino]benzenesulphonamide), 1-[4-(N-2-methoxybenzoylsulphamoyl)-phenyl]-3,3-dimethylurea, 1-[4-(N-4,5-dimethylbenzoylsulphamoyl)phenyl]-3-methylurea, 1-[4-(N-naphthylsulphamoyl)phenyl]-3,3-dimethylurea, N-(2-methoxy-5-methylbenzoyl)-4-(cyclopropylaminocarbonyl)benzenesulphonamide,
   and/or one of the following compounds, defined by general formulae,
   of the general formula (IIa) 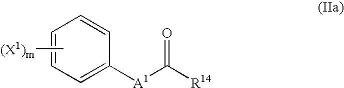
   or of the general formula (IIb) 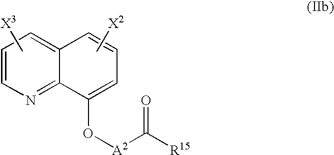
   or of the formula (IIc) 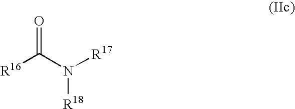
   where
m represents a number 0, 1, 2, 3, 4 or 5,
A¹ represents one of the divalent heterocyclic groupings shown below 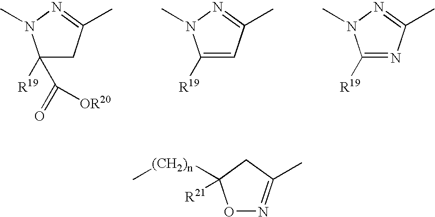
n represents a number between 1, 2, 3, 4 or 5,
A² represents optionally C₁-C₄-alkyl- and/or C₁-C₄-alkoxy-carbonyl- and/or C₁-C₄-alkenyloxy-carbonyl-substituted alkanediyl having 1 or 2 carbon atoms,
R¹⁴ represents hydroxyl, mercapto, amino, C₁-C₆-alkoxy, C₁-C₆-alkylthio, C₁-C₆-alkylamino or di-(C₁-C₄-alkyl)-amino,
R¹⁵ represents hydroxyl, mercapto, amino, C₁-C₇-alkoxy, C₁-C₆-alkenyloxy, C₁-C₆-alkenyloxy-C₁-C₆-alkoxy, C₁-C₆-alkylthio, C₁-C₆-alkylamino or di-(C₁-C₄-alkyl)-amino,
R¹⁶ represents in each case optionally fluorine-, chlorine- and/or bromine-substituted C₁-C₄-alkyl,
R¹⁷ represents hydrogen, in each case optionally fluorine-, chlorine- and/or bromine-substituted C₁-C₆-alkyl, C₂-C₆-alkenyl or C₂-C₆-alkynyl, C₁-C₄-alkoxy-C₁-C₄-alkyl, dioxolanyl-C₁-C₄-alkyl, furyl, furyl-C₁-C₄-alkyl, thienyl, thiazolyl, piperidinyl, or optionally fluorine-, chlorine- and/or bromine- or C₁-C₄-alkyl-substituted phenyl,
R¹⁸ represents hydrogen, in each case optionally fluorine-, chlorine- and/or bromine-substituted C₁-C₆-alkyl, C₂-C₆-alkenyl or C₂-C₆-alkynyl, C₁-C₄-alkoxy-C₁-C₄-alkyl, dioxolanyl-C₁-C₄-alkyl, furyl, furyl-C₁-C₄-alkyl, thienyl, thiazolyl, piperidinyl, or optionally fluorine-, chlorine- and/or bromine- or C₁-C₄-allyl-substituted phenyl,
R¹⁷ and R¹⁸ also together represent C₃-C₆-alkanediyl or C₂-C₅-oxaalkanediyl, each of which is optionally substituted by C₁-C₄-alkyl, phenyl, furyl, a fused benzene ring or by two substituents which, together with the C atom to which they are attached, form a 5- or 6-membered carboxycle,
R¹⁹ represents hydrogen, cyano, halogen, or represents in each case optionally fluorine-, chlorine- and/or bromine-substituted C₁-C₄-alkyl, C₃-C₆-cycloalkyl or phenyl,
R²⁰ represents hydrogen, optionally hydroxyl-, cyano-, halogen- or C₁-C₄-alkoxy-substituted C₁-C₆-alkyl, C₃-C₆-cycloalkyl or tri-(C₁-C₄-alkyl)-silyl,
R²¹ represents hydrogen, cyano, halogen, or represents in each case optionally fluorine-, chlorine- and/or bromine-substituted C₁-C₄-alkyl, C₃-C₆-cycloalkyl or phenyl,
X¹ represents nitro, cyano, halogen, C₁-C₄-alkyl, C₁-C₄-haloalkyl, C₁-C₄-alkoxy or C₁-C₄-haloalkoxy,
X² represents hydrogen, cyano, nitro, halogen, C₁-C₄-alkyl, C₁-C₄-haloalkyl, C₁-C₄-alkoxy or C₁-C₄-haloalkoxy,
X³ represents hydrogen, cyano, nitro, halogen, C₁-C₄-alkyl, C₁-C₄-haloalkyl, C₁-C₄-alkoxy or C₁-C₄-haloalkoxy,
   and/or the following compounds, defined by general formulae,
   of the general formula (IId) 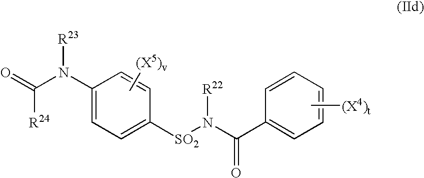
   or of the general formula (IIe) 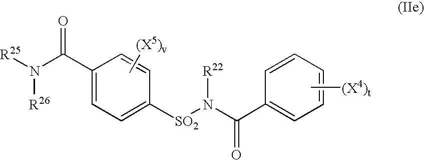
   where
t represents a number between 0 and 5,
v represents a number between 0 and 5,
R²² represents hydrogen or C₁-C₄-alkyl,
R²³ represents hydrogen or C₁-C₄-alkyl,
R²⁴ represents hydrogen, in each case optionally cyano-, halogen- or C₁-C₄-alkoxy-substituted C₁-C₆-alkyl, C₁-C₆-alkoxy, C₁-C₆-alkylthio, C₁-C₆-alkylamino or di-(C₁-C₄-alkyl)-amino, or in each case optionally cyano-, halogen- or C₁-C₄-alkyl-substituted C₃-C₆-cycloalkyl, C₃-C₆-cycloalkyloxy, C₃-C₆-cycloalkylthio or C₃-C₆-cycloalkylamino,
R²⁵ represents hydrogen, optionally cyano-, hydroxyl-, halogen- or C₁-C₄-alkoxy-substituted C₁-C₆-alkyl, in each case optionally cyano- or halogen-substituted C₃-C₆-alkenyl or C₃-C₆-alkynyl, or optionally cyano-, halogen- or C₁-C₄-alkyl-substituted C₃-C₆-cycloalkyl,
R²⁶ represents hydrogen, optionally cyano-, hydroxyl-, halogen- or C₁-C₄-alkoxy-substituted C₁-C₆-alkyl, in each case optionally cyano- or halogen-substituted C₃-C₆-alkenyl or C₃-C₆-alkynyl, optionally cyano-, halogen- or C₁-C₄-alkyl-substituted C₃-C₆-cycloalkyl, or optionally nitro-, cyano-, halogen-, C₁-C₄-alkyl-, C₁-C₄-haloalkyl-, C₁-C₄-alkoxy- or C₁-C₄-haloalkoxy-substituted phenyl, or together with R²⁵ represents in each case optionally C₁-C₄-alkyl-substituted C₂-C₆-alkanediyl or C₂-C₅-oxaalkanediyl,
X⁴ represents nitro, cyano, carboxyl, carbamoyl, formyl, sulphamoyl, hydroxyl, amino, halogen, C₁-C₄-alkyl, C₁-C₄-haloalkyl, C₁-C₄-alkoxy or C₁-C₄-haloalkoxy, and
X⁵ represents nitro, cyano, carboxyl, carbamoyl, formyl, sulphamoyl, hydroxyl, amino, halogen, C₁-C₄-alkyl, C₁-C₄-haloalkyl, C₁-C₄-alkoxy or C₁-C₄-haloalkoxy.

The formulae (I-1) and/or (I-2) provide a general definition of the compounds according to the invention. Preferred substituents or ranges of the radicals listed in the formulae given above and below are elucidated below:
W preferably represents hydrogen, C₁-C₆-alkyl, C₂-C₆-alkenyl, ethynyl, fluorine, chlorine, bromine, C₁-C₄-haloalkyl, C₁-C₆-alkoxy or C₁-C₄-haloalkoxy,
X preferably represents fluorine, chlorine, bromine, C₁-C₆-alkyl, C₁-C₄-haloalkyl, C₁-C₆-alkoxy, C₂-C₆-alkenyl, ethynyl, C₁-C₄-haloalkoxy, nitro or cyano,
Y preferably represents hydrogen, fluorine, chlorine, bromine, C₁-C₆-alkyl, C₁-C₄-haloalkyl, C₁-C₆-alkoxy, C₁-C₄-haloalkoxy or cyano,
Z preferably represents hydrogen, C₁-C₆-alkyl, fluorine, chlorine, bromine, C₁-C₆-alkoxy, C₂-C₄-alkenyl, ethynyl or represents one of the radicals 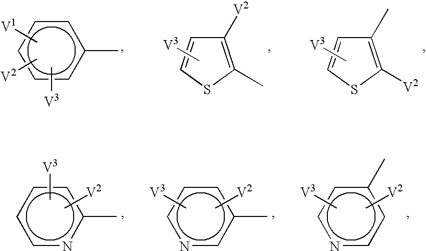
V¹ preferably represents hydrogen, halogen, C₁-C₁₂-alkyl, C₁-C₆-alkoxy, C₁-C₆-alkylthio, C₁-C₄-haloalkyl, C₁-C₄-haloalkoxy, nitro or cyano,
V² preferably represents hydrogen, fluorine, chlorine, C₁-C₆-alkyl, C₁-C₆-alkoxy, C₁-C₄-haloalkyl or C₁-C₄-haloalkoxy,
V³ preferably represents hydrogen, fluorine, chlorine, methyl or methoxy,
with the first proviso that W, X and Y do not represent bromine, C₂-C₆-alkenyl and ethynyl if Z represents V¹-, V²- and V³-substituted phenyl or hetaryl, and that, secondly, only a maximum of two of the radicals W, X and Z may represent C₂-C₆-alkenyl or ethynyl, with the proviso that in that case none of the other radicals W, X, Y and Z may represent bromine,
A preferably represents hydrogen or in each case optionally halogen-substituted C₁-C₁₂-alkyl, C₃-C₈-alkenyl, C₁-C₁₀-alkoxy-C₁-C₈-alkyl, poly-C₁-C₈-alkoxy-C₁-C₈-alkyl or C₁-C₁₀-alkylthio-C₁-C₆-alkyl, optionally halogen-, C₁-C₆-alkyl- or C₁-C₆-alkoxy-substituted C₃-C₈-cycloalkyl, in which optionally one or two ring members that are not directly adjacent has or have been replaced by oxygen and/or sulphur,
B preferably represents hydrogen, C₁-C₁₂-alkyl, or C₁-C₈-alkoxy-C₁-C₆-alkyl, or
A, B and the carbon atom to which they are attached preferably represent saturated C₃-C₁₀-cycloalkyl or unsaturated C₅-C₁₀-cycloalkyl, in which optionally a ring member has been replaced by oxygen or sulphur and which are optionally substituted once or twice by C₁-C₈-alkyl, C₃-C₁₀-cycloalkyl, C₁-C₈-haloalkyl, C₁-C₈-alkoxy, C₁-C₈-alkylthio, halogen or phenyl,
D preferably represents hydrogen, in each case optionally halogen-substituted C₁-C₁₂-alkyl, C₃-C₈-alkenyl, C₁-C₁₀-alkoxy-C₂-C₈-alkyl, poly-C₁-C₈-alkoxy-C₂-C₈-alkyl or C₁-C₁₀-alkylthio-C₂-C₈-alkyl, optionally halogen-, C₁-C₄-alkyl-, C₁-C₄-alkoxy- or C₁-C₄-haloalkyl-substituted C₄-C₈-cycloalkyl, in which optionally a ring member has been replaced by oxygen or sulphur,
G preferably represents halogen or nitro,
A and Q¹ together preferably represent C₃-C₆-alkanediyl which is in each case optionally substituted once or twice by identical or different substituents selected from C₁-C₄-alkyl and C₁-C₄-alkoxy and which may optionally be interrupted by oxygen or sulphur,
Q¹ preferably represents hydrogen, hydroxyl, C₁-C₆-alkyl, C₁-C₆-alkoxy, C₁-C₆-alkoxy-C₁-C₂-alkyl or C₁-C₆-alkylacyloxy, optionally fluorine-, chlorine-, C₁-C₄-alkyl-, C₁-C₂-haloalkyl- or C₁-C₄-alkoxy-substituted C₃-C₈-cycloalkyl, in which optionally a methylene group has been replaced by oxygen or sulphur, or phenyl optionally substituted by halogen, C₁-C₄-alkyl, C₁-C₄-alkoxy, C₁-C₂-haloalkyl, C₁-C₂-haloalkoxy, cyano or nitro,
Q² preferably represents hydrogen or C₁-C₄-alkyl, or
Q¹ and Q², together with the carbon atom to which they are attached, preferably represent optionally C₁-C₆-alkyl-, C₁-C₆-alkoxy- or C₁-C₂-haloalkyl-substituted C₃-C₇-cycloalkyl, in which optionally a ring member has been replaced by oxygen or sulphur.

In the radical definitions mentioned as being preferred, halogen, including halogen as a substituent, such as in haloalkyl, for example, represents fluorine, chlorine, bromine and iodine, especially represents fluorine and chlorine.
W with particular preference represents hydrogen, C₁-C₄-alkyl, C₂-C₃-alkenyl, ethynyl, fluorine, chlorine, bromine, trifluoromethyl or C₁-C₄-alkoxy,
X with particular preference represents fluorine, chlorine, bromine, C₁-C₄-alkyl, C₁-C₄-alkoxy, C₂-C₃-alkenyl, ethynyl, C₁-C₂-haloalkyl, C₁-C₂-haloalkoxy or cyano,
Y with particular preference represents hydrogen, fluorine, chlorine, bromine, C₁-C₄-alkyl, C₁-C₂-haloalkyl, C₁-C₄-alkoxy or C₁-C₂-haloalkoxy,
Z with particular preference represents hydrogen, C₁-C₄-alkyl, fluorine, chlorine, bromine, C₁-C₄-alkoxy, C₂-C₃-alkenyl, ethynyl, or represents the radical 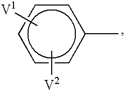
V¹ with particular preference represents hydrogen, fluorine, chlorine, bromine, C₁-C₆-alkyl, C₁-C₄-alkoxy, C₁-C₄-alkylthio, C₁-C₂-haloalkyl or C₁-C₂-haloalkoxy,
V² with particular preference represents hydrogen, fluorine, chlorine, C₁-C₄-alkyl, C₁-C₄-alkoxy, C₁-C₂-haloalkyl or C₁-C₂-haloalkoxy, with the first proviso that W, X and Y do not represent bromine, C₂-C₃-alkenyl and ethynyl if Z represents V¹- and V²-substituted phenyl, and that, secondly, only a maximum of two of the radicals W, X and Z may represent C₂-C₃-alkenyl or ethynyl, with the proviso that in that case none of the other radicals W, X, Y and Z may represent bromine,
A preferably represents hydrogen, C₁-C₁₀-alkyl or C₁-C₈-alkoxy-C₁-C₆-alkyl, the latter two of which are each optionally substituted one to three times by fluorine or chlorine, or C₃-C₇-cycloalkyl optionally substituted once to twice by fluorine, chlorine, C₁-C₄-alkyl or C₁-C₄-alkoxy, and in which optionally a ring member has been replaced by oxygen and/or sulphur,
B preferably represents hydrogen or C₁-C₆-alkyl or
A, B and the carbon atom to which they are attached with particular preference represent saturated or unsaturated C₃-C₇-cycloalkyl, in which optionally a ring member has been replaced by oxygen or sulphur, and which is optionally monosubstituted by C₁-C₆-alkyl, C₁-C₃-haloalkyl or C₁-C₆-alkoxy,
D preferably represents hydrogen, represents C₁-C₁₀-alkyl, C₃-C₆-alkenyl, C₁-C₄-alkoxy-C₂-C₄-alkyl or C₁-C₄-alkylthio-C₂-C₄-alkyl, each of which is optionally substituted one to three times by fluorine or chlorine, or represents C₄-C₇-cycloalkyl which is optionally substituted by fluorine, chlorine, C₁-C₄-alkyl, C₁-C₄-alkoxy or C₁-C₄-haloalkyl, and in which optionally a methylene group has been replaced by oxygen or sulphur,
G preferably represents bromine, chlorine or nitro,
A and Q¹ together with particular preference represent C₃-C₄-alkanediyl optionally monosubstituted by C₁-C₂-alkyl,
Q¹ with particular preference represents hydrogen, C₁-C₄-alkyl, C₁-C₄-alkoxy or C₁-C₄-alkoxy-C₁-C₂-alkyl or C₃-C₆-cycloalkyl which is optionally monosubstituted by methyl or methoxy, and in which optionally a methylene group has been replaced by oxygen,
Q² with particular preference represents hydrogen, methyl or ethyl or
Q¹ and Q² with particular preference, together with the carbon to which they are attached, represent saturated C₅-C₆-cycloalkyl which is optionally monosubstituted by C₁-C₄-alkyl or C₁-C₄-alkoxy, and in which optionally a ring member has been replaced by oxygen.

In the radical definitions mentioned as being particularly preferred, halogen, both alone and as a substituent, such as in haloalkyl, for example, represents fluorine, chlorine, bromine and iodine, particularly represents fluorine and chlorine, and especially represents fluorine.

W with very particular preference represents hydrogen, fluorine, chlorine, bromine, methyl, ethyl, n-propyl or methoxy,
X with very particular preference represents fluorine, chlorine, bromine, methyl, ethyl, n-propyl, isopropyl, methoxy, ethoxy, n-propoxy, trifluoromethyl, difluoromethoxy, trifluoromethoxy or cyano (in particular represents chlorine, bromine, methyl, ethyl, n-propyl, methoxy, ethoxy or trifluoromethyl),
Y with very particular preference represents hydrogen, fluorine, chlorine, bromine, methyl, ethyl, methoxy or trifluoromethyl (in particular represents hydrogen, chlorine, bromine or methyl),
Z with very particular preference represents hydrogen, methyl, ethyl, n-propyl, isopropyl, fluorine, chlorine, bromine, methoxy or represents the radical 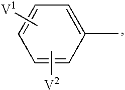
V₁ with very particular preference represents hydrogen, fluorine, chlorine, bromine, methyl, ethyl, n-propyl, isopropyl, n-butyl, isobutyl, tert-butyl, methoxy, ethoxy, n-propoxy, isopropoxy, trifluoromethyl or trifluoromethoxy,
V² with very particular preference represents hydrogen, fluorine, chlorine, methyl, methoxy or trifluoromethyl,
A with very particular preference represents hydrogen, C₁-C₄-alkyl or C₁-C₄-alkoxy-C₁-C₂-alkyl, C₃-C₆-cycloalkyl which is optionally monosubstituted by fluorine, methyl, ethyl or methoxy, and in which optionally a ring member has been replaced by oxygen and/or sulphur,
B with very particular preference represents hydrogen, methyl or ethyl, or
A, B and the carbon atom to which they are attached with very particular preference represent saturated C₃-C₆-cycloalkyl in which optionally a ring member has been replaced by oxygen and which is optionally monosubstituted by methyl, ethyl, trifluoromethyl, methoxy, ethoxy, n-propoxy, n-butoxy or isobutoxy,
D with very particular preference represents hydrogen or represents C₁-C₈-alkyl, C₃-C₄-alkenyl, C₁-C₆-alkoxy-C₂-C₄-alkyl, C₁-C₄-alkylthio-C₂-C₄-alkyl or C₄-C₆-cycloalkyl, each of which is optionally substituted one to three times by fluorine,
G with very particular preference represents chlorine or nitro,
A and Q¹ with very particular preference together represent C₃-C₄-alkanediyl optionally monosubstituted by methyl,
Q¹ with very particular preference represents hydrogen, methyl, ethyl, n-propyl, isopropyl, methoxy, ethoxy, n-propoxy, cyclopropyl, cyclopentyl or cyclohexyl,
Q² with very particular preference represents hydrogen, methyl or ethyl, or
Q¹ and Q² with very particular preference, together with the carbon to which they are attached, represent saturated C₅-C₆-cycloalkyl which is optionally substituted by methyl, ethyl, n-propyl, isopropyl, methoxy, ethoxy, propoxy or butoxy, and in which optionally a ring member has been replaced by oxygen.
W most preferably represents hydrogen, chlorine, methyl or ethyl.
X most preferably represents chlorine, bromine, methyl or ethyl,
Y most preferably represents hydrogen, chlorine, bromine or methyl,
Z most preferably represents methyl or represents the radical 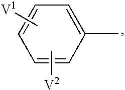
V¹ most preferably represents hydrogen, fluorine, chlorine or trifluoromethyl,
V² most preferably represents hydrogen, fluorine, chlorine or trifluoromethyl,
A most preferably represents C₁-C₄-alkyl,
B most preferably represents methyl,
A, B and the carbon atom to which they are attached most preferably represent saturated C₃-C₆-cycloalkyl in which optionally a ring member has been replaced by oxygen,
D most preferably represents methyl,
G most preferably represents chlorine,
A and Q¹ together most preferably represent C₃-C₄-alkanediyl,
Q¹ most preferably represents hydrogen or methyl,
Q² most preferably represents hydrogen or methyl.

The general radical definitions and/or elucidations set out above, or those set out in ranges of preference, can be combined with one another arbitrarily, i.e. including combinations between the respective ranges and ranges of preference. They apply to the end products and also to the precursors and intermediates accordingly.

Preference in accordance with the invention is given to the compounds of the formula (I) in which there is a combination of the definitions set out above as being preferred (preferable).

Particular preference in accordance with the invention is given to the compounds of the formula (I) in which there is a combination of the definitions set out above as being particularly preferred.

Very particular preference in accordance with the invention is given to the compounds of the formula (I) in which there is a combination of the definitions set out above as being very particularly preferred.

Most preferred in accordance with the invention are the compounds of the formula (I) in which there is a combination of the definitions set out above as being most preferred.

Saturated or unsaturated hydrocarbon radicals such as alkyl or alkenyl, both alone and in conjunction with heteroatoms, such as in alkoxy, for example, can as far as possible be straight-chain or branched in each case.

Optionally substituted radicals can be substituted one or more times unless indicated otherwise, and in the case of multiple substitutions the substituents can be identical or different.

Preferred definitions of the groups listed above in connection with the crop plant tolerance promoter compounds (“herbicide safeners”) of the formulae (IIa), (IIb), (IIc), (IId) and (IIe) are defined below.
m preferably represents the numbers 0, 1, 2, 3 or 4.
A¹ preferably represents one of the divalent heterocyclic groupings shown below 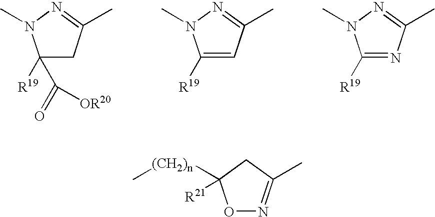
n preferably represents the numbers 0, 1, 2, 3 or 4.
A² preferably represents in each case optionally methyl-, ethyl-, methoxycarbonyl- or ethoxycarbonyl- or allyloxycarbonyl-substituted methylene or ethylene.
R¹⁴ preferably represents hydroxyl, mercapto, amino, methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, dimethylamino or diethylamino.
R¹⁵ preferably represents hydroxyl, mercapto, amino, methoxy, ethoxy, n- or i-propoxy, n-, i-, s- or t-butoxy, 1-methylhexyloxy, allyloxy, 1-allyloxymethylethoxy, methylthio, ethylthio, n- or i-propylthio, n-, i-, s- or t-butylthio, methylamino, ethylamino, n- or i-propylamino, n-, i-, s- or t-butylamino, dimethylamino or diethylamino.
R¹⁶ preferably represents in each case optionally fluorine-, chlorine-, and/or bromine-substituted methyl, ethyl, n- or i-propyl.
R¹⁷ preferably represents hydrogen, in each case optionally fluorine- and/or chlorine-substituted methyl, ethyl, n- or i-propyl, n-, i-, s- or t-butyl, propenyl, butenyl, propynyl or butynyl, methoxymethyl, ethoxymethyl, methoxyethyl, ethoxyethyl, dioxolanylmethyl, furyl, furylmethyl, thienyl, thiazolyl, piperidinyl, or optionally fluorine-, chlorine-, methyl-, ethyl-, n- or i-propyl-, n-, i-, s- or t-butyl-substituted phenyl.
R¹⁸ preferably represents hydrogen, in each case optionally fluorine- and/or chlorine-substituted methyl, ethyl, n- or i-propyl, n-, i-, s- or t-butyl, propenyl, butenyl, propynyl or butynyl, methoxymethyl, ethoxymethyl, methoxyethyl, ethoxyethyl, dioxolanylmethyl, furyl, furylmethyl, thienyl, thiazolyl, piperidinyl, or optionally fluorine-, chlorine-, methyl-, ethyl-, n- or i-propyl-, n-, i-, s- or t-butyl-substituted phenyl, or together with R¹⁷ represents one of the radicals —CH₂—O—CH₂—CH₂— and —CH₂—CH₂—O—CH₂—CH₂— which are optionally substituted by methyl, ethyl, furyl, phenyl, a fused benzene ring or by two substituents which, together with the C atom to which they are attached, form a 5- or 6-membered carbocycle.
R¹⁹ preferably represents hydrogen, cyano, fluorine, chlorine, bromine, or represents in each case optionally fluorine-, chlorine- and/or bromine-substituted methyl, ethyl, n- or i-propyl, cyclopropyl, cyclobutyl, cyclopentyl, cyclohexyl or phenyl.
R²⁰ preferably represents hydrogen, optionally hydroxyl-, cyano-, fluorine-, chlorine-, methoxy-, ethoxy-, n- or i-propoxy-substituted methyl, ethyl, n- or i-propyl, n-, i-, s- or t-butyl.
R²¹ preferably represents hydrogen, cyano, fluorine, chlorine, bromine, or represents in each case optionally fluorine-, chlorine- and/or bromine-substituted methyl, ethyl, n- or i-propyl, n-, i-, s- or t-butyl, cyclopropyl, cyclobutyl, cyclopentyl, cyclohexyl or phenyl.
X¹ preferably represents nitro, cyano, fluorine, chlorine, bromine, methyl, ethyl, n- or i-propyl, n-, i-, s- or t-butyl, difluoromethyl, dichloromethyl, trifluoromethyl, trichloromethyl, chlorodifluoromethyl, fluorodichloromethyl, methoxy, ethoxy, n- or i-propoxy, difluoromethoxy or trifluoromethoxy.
X² preferably represents hydrogen, nitro, cyano, fluorine, chlorine, bromine, methyl, ethyl, n- or i-propyl, n-, i-, s- or t-butyl, difluoromethyl, dichloromethyl, trifluoromethyl, trichloromethyl, chlorodifluoromethyl, fluorodichloromethyl, methoxy, ethoxy, n- or i-propoxy, difluoromethoxy or trifluoromethoxy.
X³ preferably represents hydrogen, nitro, cyano, fluorine, chlorine, bromine, methyl, ethyl, n- or i-propyl, n-, i-, s- or t-butyl, difluoromethyl, dichloromethyl, trifluoromethyl, trichloromethyl, chlorodifluoromethyl, fluorodichloromethyl, methoxy, ethoxy, n- or i-propoxy, difluoromethoxy or trifluoromethoxy.
v preferably represents the numbers 0, 1, 2, 3 or 4.
t preferably represents the numbers 0, 1, 2, 3 or 4.
R²² preferably represents hydrogen, methyl, ethyl, n- or i-propyl.
R²³ preferably represents hydrogen, methyl, ethyl, n- or i-propyl.
R²⁴ preferably represents hydrogen, in each case optionally cyano-, fluorine-, chlorine-, methoxy-, ethoxy-, n- or i-propoxy-substituted methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, dimethylamino or diethylamino, or in each case optionally cyano-, fluorine-, chlorine-, bromine-, methyl-, ethyl-, n- or i-propyl-substituted cyclopropyl, cyclobutyl, cyclopentyl, cyclohexyl, cyclopropyloxy, cyclobutyloxy, cyclopentyloxy, cyclohexyloxy, cyclopropylthio, cyclobutylthio, cyclopentylthio, cyclohexylthio, cyclopropylamino, cyclobutylamino, cyclopentylamino or cyclohexylamino.
R²⁵ preferably represents hydrogen, in each case optionally cyano-, hydroxyl-, fluorine-, chlorine-, methoxy-, ethoxy-, n- or i-propoxy-substituted methyl, ethyl, n- or i-propyl, n-, i- or s-butyl, in each case optionally cyano-, fluorine-, chlorine- or bromine-substituted propenyl, butenyl, propynyl or butynyl, or in each case optionally cyano-, fluorine-, chlorine-, bromine-, methyl-, ethyl-, n- or i-propyl-substituted cyclopropyl, cyclobutyl, cyclopentyl or cyclohexyl.
R²⁶ preferably represents hydrogen, in each case optionally cyano-, hydroxyl-, fluorine-, chlorine-, methoxy-, ethoxy-, n- or i-propoxy-substituted methyl, ethyl, n- or i-propyl, n-, i- or s-butyl, in each case optionally cyano-, fluorine-, chlorine- or bromine-substituted propenyl, butenyl, propynyl or butynyl, in each case optionally cyano-, fluorine-, chlorine-, bromine-, methyl-, ethyl-, n- or i-propyl-substituted cyclopropyl, cyclobutyl, cyclopentyl or cyclohexyl, or optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, difluoromethoxy- or trifluoromethoxy-substituted phenyl, or together with R²⁵ represents in each case optionally methyl- or ethyl-substituted butane-1,4-diyl (trimethylene), pentane-1,5-diyl, 1-oxabutane-1,4-diyl or 3-oxapentane-1,5-diyl.
X⁴ preferably represents nitro, cyano, carboxyl, carbamoyl, formyl, sulphamoyl, hydroxyl, amino, fluorine, chlorine, bromine, methyl, ethyl, n- or i-propyl, n-, i-, s- or t-butyl, trifluoromethyl, methoxy, ethoxy, n- or i-propoxy, difluoro-methoxy or trifluoromethoxy.
X⁵ preferably represents nitro, cyano, carboxyl, carbamoyl, formyl, sulphamoyl, hydroxyl, amino, fluorine, chlorine, bromine, methyl, ethyl, n- or i-propyl, n-, i-, s- or t-butyl, trifluoromethyl, methoxy, ethoxy, n- or i-propoxy, difluoro-methoxy or trifluoromethoxy.

Examples of the compounds of the formula (IIa) which are very particularly preferred as herbicide safeners according to the invention are listed in Table 1 below.

TABLE 1
Examples of the compounds of the formula (IIa)

| | |
|---|---|
| | (IIa) |
| 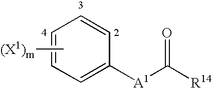 | |

| Example | (Positions) | | |
|---|---|---|---|
| No. | (X¹)ₘ | A¹ | R¹⁴ |
| IIa-1 | (2) Cl, (4) Cl | 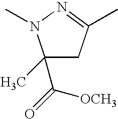 | OCH₃ |
| IIa-2 | (2) Cl, (4) Cl | 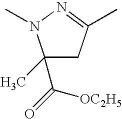 | OCH₃ |
| IIa-3 | (2) Cl, (4) Cl | 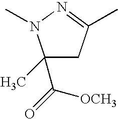 | OC₂H₅ |
| IIa-4 | (2) Cl, (4) Cl | 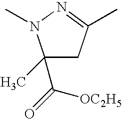 | OC₂H₅ |
| IIa-5 | (2) Cl | 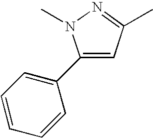 | OCH₃ |
| IIa-6 | (2) Cl, (4) Cl | 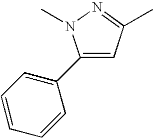 | OCH₃ |
| IIa-7 | (2) F | 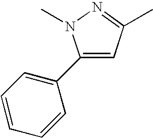 | OCH₃ |
| IIa-8 | (2) F | 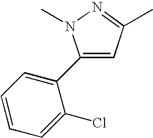 | OCH₃ |
| IIa-9 | (2) Cl, (4) Cl | 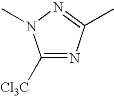 | OC₂H₅ |
| IIa-10 | (2) Cl, (4) CF₃ | 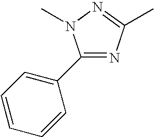 | OCH₃ |
| IIa-11 | (2) Cl | 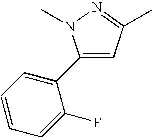 | OCH₃ |
| IIa-12 | — | 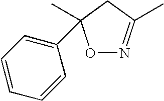 | OC₂H₅ |
| IIa-13 | (2) Cl, (4) Cl | 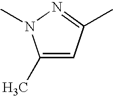 | OC₂H₅ |
| IIa-14 | (2) Cl, (4) Cl | 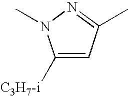 | OC₂H₅ |
| IIa-15 | (2) Cl, (4) Cl | 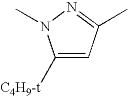 | OC₂H₅ |
| IIa-16 | (2) Cl, (4) Cl | 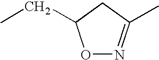 | OC₂H₅ |
| IIa-17 | (2) Cl, (4) Cl | 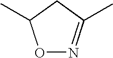 | OC₂H₅ |
| IIa-18 | — | 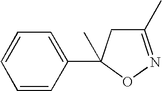 | OH |

Examples of the compounds of the formula (IIb) which are very particularly preferred as herbicide safeners according to the invention are listed in Table 2 below.

TABLE 2
Examples of the compounds of the formula (IIb)

| | |
|---|---|
| | (IIb) |
| 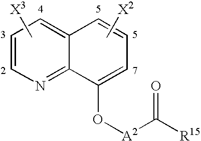 | |

| Ex- | | | | |
|---|---|---|---|---|
| ample | (Position) | (Position) | | |
| No. | X² | X³ | A² | R¹⁵ |
| IIb-I | (5) | — | CH₂ | OH |
| | Cl | | | |
| IIb-2 | (5) | — | CH₂ | OCH₃ |
| | Cl | | | |
| IIb-3 | (5) | — | CH₂ | OC₂H₅ |
| | Cl | | | |
| IIb-4 | (5) | — | CH₂ | OC₃H₇-n |
| | Cl | | | |
| IIb-5 | (5) | — | CH₂ | OC₃H₇-i |
| | Cl | | | |
| IIb-6 | (5) | — | CH₂ | OC₄H₉-n |
| | Cl | | | |
| IIb-7 | (5) | — | CH₂ | OCH(CH₃)C₅H₁₁-n |
| | Cl | | | |
| IIb-8 | (5) | (2) | CH₂ | OH |
| | Cl | F | | |
| IIb-9 | (5) | (2) | CH₂ | OH |
| | Cl | Cl | | |
| IIb-10 | (5) | — | CH₂ | OCH₂CH═CH₂ |
| | Cl | | | |
| IIb-11 | (5) | — | CH₂ | OC₄H₉-i |
| | Cl | | | |
| IIb-12 | (5) Cl | — | CH₂ | 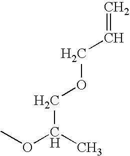 |
| IIb-13 | (5) Cl | — | 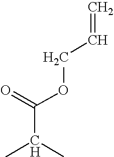 | OCH₂CH═CH₂ |
| IIb-14 | (5) Cl | — | 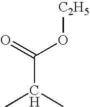 | OC₂H₅ |
| IIb-15 | (5) Cl | — | 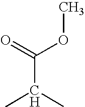 | OCH₃ |

Examples of the compounds of the formula (IIc) which are very particularly preferred as herbicide safeners according to the invention are listed in Table 3 below.

TABLE 3
Examples of the compounds of the formula (IIc)

| | |
|---|---|
| | (IIc) |
| 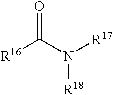 | |

| Example | | |
|---|---|---|
| No. | R¹⁶ | N(R¹⁷, R¹⁸) |
| IIc-1 | CHCl₂ | N(CH₂CH═CH₂)₂ |
| IIc-2 | CHCl₂ | 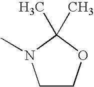 |
| IIc-3 | CHCl₂ | 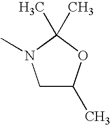 |
| IIc-4 | CHCl₂ | 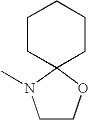 |
| IIc-5 | CHCl₂ | 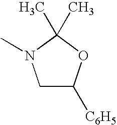 |
| IIc-6 | CHCl₂ | 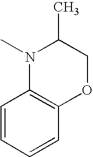 |
| IIc-7 | CHCl₂ | 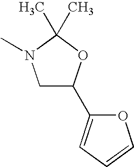 |

Examples of the compounds of the formula (IId) which are very particularly preferred as herbicide safeners according to the invention are listed in Table 4 below.

TABLE 4
Examples of the compounds of the formula (IId)

| | |
|---|---|
| | (IId) |
| 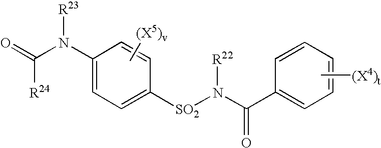 | |

| Example | | | | (Positions) | (Positions) |
|---|---|---|---|---|---|
| No. | R²² | R²³ | R²⁴ | (X⁴)ₜ | (X⁵)ᵥ |
| IId-1 | H | H | CH₃ | (2) OCH₃ | — |
| IId-2 | H | H | C₂H₅ | (2) OCH₃ | — |
| IId-3 | H | H | C₃H₇-n | (2) OCH₃ | — |
| IId-4 | H | H | C₃H₇-i | (2) OCH₃ | — |
| IId-5 | H | H | 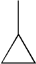 | (2) OCH₃ | — |
| IId-6 | H | H | CH₃ | (2) OCH₃ | — |
| | | | | (5) CH₃ | |
| IId-7 | H | H | C₂H₅ | (2) OCH₃ | — |
| | | | | (5) CH₃ | |
| IId-8 | H | H | C₃H₇-n | (2) OCH₃ | — |
| | | | | (5)CH₃ | |
| IId-9 | H | H | C₃H₇-i | (2) OCH₃ | — |
| | | | | (5) CH₃ | |
| IId-10 | H | H | 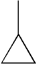 | (2) OCH₃(5) CH₃ | — |
| IId-11 | H | H | OCH₃ | (2) OCH₃ | — |
| | | | | (5) CH₃ | |
| IId-12 | H | H | OC₂H₅ | (2) OCH₃ | — |
| | | | | (5) CH₃ | |
| IId-13 | H | H | OC₃H₇-i | (2) OCH₃ | — |
| | | | | (5) CH₃ | |
| IId-14 | H | H | SCH₃ | (2) OCH₃ | — |
| | | | | (5) CH₃ | |
| IId-15 | H | H | SC₂H₅ | (2) OCH₃ | — |
| | | | | (5) CH₃ | |
| IId-16 | H | H | SC₃H₇-i | (2) OCH₃ | — |
| | | | | (5) CH₃ | |
| IId-17 | H | H | NHCH₃ | (2) OCH₃ | — |
| | | | | (5) CH₃ | |
| IId-18 | H | H | NHC₂H₅ | (2) OCH₃ | — |
| | | | | (5) CH₃ | |
| IId-19 | H | H | NHC₃H₇-i | (2) OCH₃ | — |
| | | | | (5) CH₃ | |
| IId-20 | H | H | 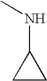 | (2) OCH₃(5) CH₃ | — |
| IId-21 | H | H | NHCH₃ | (2) OCH₃ | — |
| IId-22 | H | H | NHC₃H₇-i | (2) OCH₃ | — |
| IId-23 | H | H | N(CH₃)₂ | (2) OCH₃ | — |
| IId-24 | H | H | N(CH₃)₂ | (3) CH₃ | — |
| | | | | (4) CH₃ | |
| IId-25 | H | H | CH₂—O—CH₃ | (2) OCH₃ | — |

Examples of the compounds of the formula (IIe) which are very particularly preferred as herbicide safeners according to the invention are listed in Table 5 below.

TABLE 5
Examples of the compounds of the formula (IIe)

| | |
|---|---|
| | (IIe) |
| 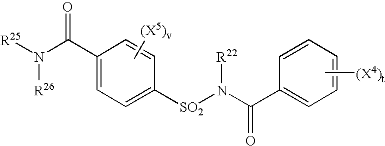 | |

| Example | | | | (Positions) | (Positions) |
|---|---|---|---|---|---|
| No. | R²² | R²⁵ | R²⁶ | (X⁴)ₜ | (X⁵)ᵥ |
| IIe-1 | H | H | CH₃ | (2) OCH₃ | — |
| IIe-2 | H | H | C₂H₅ | (2) OCH₃ | — |
| IIe-3 | H | H | C₃H₇-n | (2) OCH₃ | — |
| IIe-4 | H | H | C₃H₇-i | (2) OCH₃ | — |
| IIe-5 | H | H | 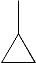 | (2) OCH₃ | — |
| IIe-6 | H | CH₃ | CH₃ | (2) OCH₃ | — |
| IIe-7 | H | H | CH₃ | (2) OCH₃ | — |
| | | | | (5) CH₃ | |
| IIe-8 | H | H | C₂H₅ | (2) OCH₃ | — |
| | | | | (5) CH₃ | |
| IIe-9 | H | H | C₃H₇-n | (2) OCH₃ | — |
| | | | | (5) CH₃ | |
| IIe-10 | H | H | C₃H₇-i | (2) OCH₃ | — |
| | | | | (5) CH₃ | |
| IIe-11 | H | H | 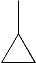 | (2) OCH₃(5) CH₃ | — |
| IIe-12 | H | CH₃ | CH₃ | (2) OCH₃ | — |
| | | | | (5) CH₃ | |

Most preferred as crop plant tolerance promoter compound [component (b′)] are cloquintocet-mexyl, fenchlorazole-ethyl, isoxadifen-ethyl, mefenpyr-diethyl, furilazole, fenclorim, cumyluron, dymron, dimepiperate and the compounds IIe-5 and IIe-11, and particular emphasis is given to cloquintocet-mexyl and mefenpyr-diethyl.

The compounds of the general formula (IIa) to be used as safeners according to the invention are known and/or can be prepared by processes known per se (cf. WO-A-91/07874, WO-A-95/07897).

The compounds of the general formula (IIb) to be used as safeners according to the invention are known and/or can be prepared by processes known per se (cf. EP-A-191736).

The compounds of the general formula (IIc) to be used as safeners according to the invention are known and/or can be prepared by processes known per se (cf. DE-A-2218097, DE-A-2350547).

The compounds of the general formula (IId) to be used as safeners according to the invention are known and/or can be prepared by processes known per se (cf. DE-A-19621522/U.S. Pat. No. 6,235,680).

The compounds of the general formula (IIe) to be used as safeners according to the invention are known and can be prepared by processes known per se (cf. WO-A-99/667951U.S. Pat. No. 6,251,827).

Examples of the selective-herbicidal combinations according to the invention comprising in each case one active compound of the formula (I-1) or (I-2) and one of the safeners defined above are listed in Table 6 below.

TABLE 6
Examples of the combinations according to the invention

| Active compounds of the formula (I) | Safeners |
|---|---|
| I-1 | cloquintocet-mexyl |
| I-1 | fenchlorazole-ethyl |
| I-1 | isoxadifen-ethyl |
| I-1 | mefenpyr-diethyl |
| I-1 | furilazole |
| I-1 | fenclorim |
| I-1 | cumyluron |
| I-1 | daimuron/dymron |
| I-1 | dimepiperate |
| I-1 | Ile-11 |
| I-1 | Ile-5 |
| I-2 | cloquintocet-mexyl |
| I-2 | fenchlorazole-ethyl |
| I-2 | isoxadifen-ethyl |
| I-2 | mefenpyr-diethyl |
| I-2 | furilazole |
| I-2 | fenclorim |
| I-2 | cumyluron |
| I-2 | daimuron/dymron |
| I-2 | dimepiperate |
| I-2 | Ile-11 |
| I-2 | Ile-5 |

It has now surprisingly been found that the above-defined active-compound combinations of phenyl-substituted [1,2]-dioxazine derivatives of the formula (I-1) or phenyl-substituted dihydropyrone derivatives of the formula (I-2) and safeners (antidotes) from the group (b′) set out above combine very good useful plant tolerance with a high herbicidal activity and can be used in various crops, in particular in cereals (especially wheat), but also in soya, potatoes, maize and rice, for selective weed control.

In this context it is considered surprising that, from a multiplicity of known safeners or antidotes capable of antagonizing the damaging effect of a herbicide on the crop plants, it is specifically the compounds of group (b′) set out above which are suitable for compensating—almost completely—the damaging effect of substituted cyclic keto enols on the crop plants, without at the same time having any critical adverse effect on the herbicidal activity against the weeds.

Emphasis may be given here to the particularly advantageous effect of the particularly preferred and most preferred combination partners from group (b′), particularly with regard to the gentle treatment of cereal plants, such as wheat, barley and rye, for example, but also maize and rice, as crop plants.

Using, for example, according to process (A), 4-(2-methyl-5-bromo)phenyl-2,6,6-trimethyloxazine-3,5-dione or its enol as starting material, the course of the process of the invention can be represented by the following reaction scheme:

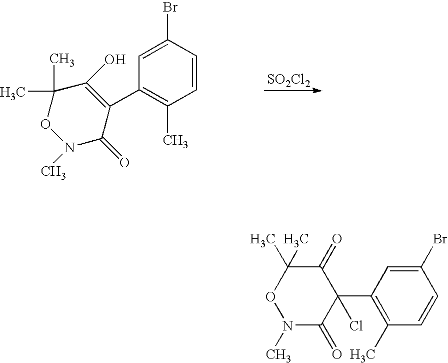

Using, for example, according to process (B), 3-[2-methyl-5-(4-chlorophenyl)phenyl]-5,5,6,6-tetramethyl-5,6-dihydropyrone-2,4-dione or its enol, the course of the process of the invention can be represented by the following reaction scheme:

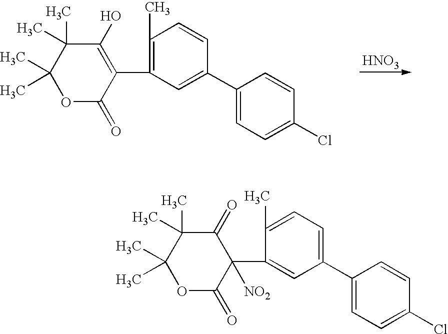

Process (A) is characterized in that compounds of the formula (II-1) or (II-2) in which A, B, D, Q¹, Q², W, X, Y and Z have the definitions indicated above are reacted in the presence of a diluent and a halogenating agent and optionally a free-radical initiator. Examples of free-radical initiators which can be used include benzoyl peroxide or azobisisobutyronitrile.

Diluents which can be used in process (A) of the invention are all inert organic solvents. With preference it is possible to use hydrocarbons such as benzene, toluene and xylene, and also ethers, such as dibutyl ether, tetrahydrofuran, dioxane, glycol dimethyl ether and diglycol dimethyl ether, and also halogenated hydrocarbons, such as dichloromethane, chloroform, carbon tetrachloride, dichloroethane, chlorobenzene, dichlorobenzene, but also esters such as ethyl acetate.

Examples of suitable halogenating agents for process A include sulphuryl chloride, sulphuryl bromide, thionyl chloride, thionyl bromide, imides such as N-bromosuccinimide, N-chlorosuccinimide, and also chlorosulphonic acid, but also hypochlorites such as tert-butyl hypochlorite, for example.

The reaction temperatures when carrying out process (A) of the invention can be varied within a relatively wide range. General operating temperatures are between −40° C. and 150° C., preferably between 0° C. and 100° C.

Process (A) of the invention is generally carried out under atmospheric pressure.

When carrying out process (A) of the invention, the reaction components of the formula (II) and the halogenating agents are generally used in approximately equimolar amounts. It is, however, also possible to use one or the other component in a relatively large excess (up to 3 mol).

Purification takes place in general after aqueous workup, by crystallization or by chromatographic purification on silica gel.

Process (B) is characterized in that compounds of the formula (II-1) or (II-2) in which A, B, D, Q¹, Q², W, X, Y, Z and m have the definitions indicated above are reacted in the presence of a diluent and in the presence of a nitrating agent.

Diluents which can be used in process (B) of the invention are all inert organic solvents. With preference it is possible to use halogenated hydrocarbons such as methylene chloride, chloroform, dichlorobenzene and dichloroethane.

Suitable nitrating agents include nitrating acids, preferably fuming nitric acid.

The reaction temperatures when carrying out process (B) of the invention can be varied within a relatively wide range. General operating temperatures are between −50° C. and 150° C., preferably between 0° C. and 80° C.

Process (B) of the invention is generally carried out under atmospheric pressure.

When carrying out process (B) of the invention the reaction components of the formula (II) and the nitrating reagent are generally used in approximately equimolar amounts. It is, however, also possible to use one or the other component in a relatively large excess (up to 5 mol).

Purification takes place after customary workup by crystallization or chromatographic purification on silica gel.

The active compounds are well tolerated by plants, have favourable homeotherm toxicity and are environmentally friendly; they are suitable for protecting plants and plant organs, for increasing harvest yields, for improving the quality of the harvested material and for controlling animal pests, in particular insects, arachnids and nematodes encountered in agriculture, in forests, in gardens and leisure facilities, in the protection of stored products and materials and in the hygiene sector. They are preferably used as crop protection agents. They are active against normally sensitive and resistant species and against all or some stages of development. The abovementioned pests include:

From the order of the Isopoda, for example, Oniscus asellus, Armadillidium vulgare and Porcellio scaber.

From the order of the Diplopoda, for example, Blaniulus guttulatus.

From the order of the Chilopoda, for example, Geophilus carpophagus and Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Acheta domesticus, Gryllotalpa spp., Locusta migratoria migratorioides, Melanoplus spp. and Schistocerca gregaria.

From the order of the Blattaria, for example, Blatta orientalis, Periplaneta americana, Leucophaea maderae and Blattella germanica.

From the order of the Dermaptera, for example, Forficula auricularia.

From the order of the Isoptera, for example, Reticulitermes spp.

From the order of the Phthiraptera, for example, Pediculus humanus corporis, Haematopinus spp., Linognathus spp., Trichodectes spp., Damalinia spp.

From the order of the Thysanoptera, for example, Hercinothrips femoralis, Thrips tabaci, Thrips palmi, Frankliniella accidentalis.

From the order of the Heteroptera, for example, Eurygaster spp., Dysdercus intermedius, Piesma quadrata, Cimex lectularius, Rhodnius prolixus and Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp. and Psylla spp.

From the order of the Lepidoptera, for example, Pectinophora gossypiella, Bupalus piniarius, Chematobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofinannophila pseudospretella, Cacoecia podana, Capua reticulana, Choristoneura fumiferana, Clysia ambiguella, Homona magnanima, Tortrix viridana, Cnaphalocerus spp. and Oulema oryzae.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica and Lissorhoptrus oryzophilus.

From the order of the Hymenoptera, for example, Diprion spp., Hoplocampa spp., Lasius spp., Monomorium pharaonis and Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp. and Liriomyza spp.

From the order of the Siphonaptera, for example, Xenopsylla cheopis and Ceratophyllus spp.

From the class of the Arachnida, for example, Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp. and Brevipalpus spp.

The plant-parasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp. and Bursaphelenchus spp.

If appropriate, the compounds according to the invention may also be used in certain concentrations or application rates to act as herbicides and microbicides, for example as fungicides, antimycotics and bactericides. If appropriate, they can also be employed as intermediates or precursors for the synthesis of further active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and recombinant methods or by combinations of these methods, including the transgenic plants and inclusive of the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offsets and seeds.

The treatment according to the invention of the plants and plant parts with the active compounds is carried out directly or by allowing the compounds to act on their surroundings, habitat or storage space by the customary treatment methods, for example by immersion, spraying, evaporation, fogging, scattering, painting on, injecting and, in the case of propagation material, in particular in the case of seeds, also by applying one or more coats.

The active compounds can be converted into the customary formulations such as solutions, emulsions, wettable powders, suspensions, powders, dusts, pastes, soluble powders, granules, suspension-emulsion concentrates, natural and synthetic materials impregnated with active compound, and microencapsulations in polymeric materials.

These formulations are produced in a known manner, for example by mixing the active compounds with extenders, that is, liquid solvents and/or solid carriers, optionally with the use of surfactants, that is, emulsifiers and/or dispersants, and/or foam formers.

If the extender used is water, it is also possible, for example, to use organic solvents as cosolvents. The following are essentially suitable as liquid solvents: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, or else water.

Suitable solid carriers are:

for example ammonium salts and ground natural minerals such as kaolins, clays, talc, chalk, quartz, attapulgite, montmorillonite or diatomaceous earth, and ground synthetic minerals such as highly disperse silica, alumina and silicates; suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks; suitable emulsifiers and/or foam formers are: for example nonionic and anionic emulsifiers such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates; suitable dispersants are: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other additives can be mineral and vegetable oils.

It is possible to use colorants such as inorganic pigments, for example iron oxide, titanium oxide and Prussian Blue, and organic colorants such as alizarin colorants, azo colorants and metal phthalocyanine colorants, and trace nutrients such as salts of iron, manganese, boron, copper, cobalt, molybdenum and zinc.

The formulations generally comprise between 0.1 and 95% by weight of active compound, preferably between 0.5 and 90%.

The active compounds according to the invention, as such or in their formulations, can also be used as a mixture with known fungicides, bactericides, acaricides, nematicides or insecticides, for example in order to widen the spectrum of action or to prevent the development of resistances in this way. In many cases, synergistic effects result, i.e. the activity of the mixture exceeds the activity of the individual components.

Compounds which are suitable as mixing partners are, for example, the following:

Fungicides:

2-phenylphenol; 8-hydroxyquinoline sulphate; acibenzolar-S-methyl; aldimorph; amidoflumet; ampropylfos; ampropylfos-potassium; andoprim; anilazine; azaconazole; azoxystrobin; benalaxyl; benodanil; benomyl; benthiavalicarb-isopropyl; benzamacril; benzamacril-isobutyl; bilanafos; binapacryl; biphenyl; bitertanol; blasticidin-S; bromuconazole; bupirimate; buthiobate; butylamine; calcium polysulphide; capsimycin; captafol; captan; carbendazim; carboxin; carpropamid; carvone; chinomethionat; chlobenthiazone; chlorfenazole; chloroneb; chlorothalonil; chlozolinate; clozylacon; cyazofamid; cyflufenamid; cymoxanil; cyproconazole; cyprodinil; cyprofuram; Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulfamide; flutolanil; flutriafol; folpet; fosetyl-A1; fosetyl-sodium; fuberidazole; furalaxyl; furametpyr; furcarbanil; furmecyclox; guazatine; hexachlorobenzene; hexaconazole; hymexazole; imazalil; imibenconazole; iminoctadine triacetate; iminoctadine tris(albesilate); iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; metconazole; methasulfocarb; methfuroxam; metiram; metominostrobin; metsulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazole; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolenitrine; quinconazole; quinoxyfen; quintozene; simeconazole; spiroxamine; sulphur; tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole; uniconazole; validamycin A; vinclozolin; zineb; ziram; zoxamide; (2S)-N-[2-[4-[[3-(4-chlorophenyl)-2-propynyl]oxy]-3-methoxyphenyl]ethyl]-3-methyl-2-[(methyl-sulphonyl)amino]butanamide; 1-(1-naphthalenyl)-1H-pyrrole-2,5-dione; 2,3,5,6-tetrachloro-4-(methylsulphonyl)pyridine; 2-amino-4-methyl-N-phenyl-5-thiazole-carboxamide; 2-chloro-N-(2,3-dihydro-1,1,3-trimethyl-1H-inden-4-yl)-3-pyridine-carboxamide; 3,4,5-trichloro-2,6-pyridinedicarbonitrile; actinovate; cis-1-(4-chlorophenyl)-2-(1H-1,2,4-triazol-1-yl)cycloheptanol; methyl 1-(2,3-dihydro-2,2-dimethyl-1H-inden-1-yl)-1H-imidazole-5-carboxylate; monopotassium carbonate; N-(6-methoxy-3-pyridinyl)cyclopropanecarboxamide; N-butyl-8-(1,1-dimethylethyl)-1-oxaspiro[4.5]decane-3-amine; sodium tetrathiocarbonate;

and copper salts and preparations, such as Bordeaux mixture; copper hydroxide; copper naphthenate; copper oxychloride; copper sulphate; cufraneb; cuprous oxide; mancopper; oxine-copper.

Bactericides:

bronopol, dichlorophen, nitrapyrin, nickel dimethyldithiocarbamate, kasugamycin, octhilinone, furancarboxylic acid, oxytetracyclin, probenazole, streptomycin, teclof-talam, copper sulphate and other copper preparations.

Insecticides/acaricides/nematicides:

Insecticides/acaricides/nematicides

1. Acetylcholine esterase (AChE) inhibitors
1.1 Carbamates, for example
   alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulfan, cloethocarb, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, trimethacarb, XMC, xylylcarb
   Triazamates
1.2 Organophosphates, for example
   acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-S-methyl, demeton-S-methylsulphon, dialifos, diazinon, di-chlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfo-thion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion
2. Sodium channel modulators/voltage-dependent sodium channel blockers
2.1 Pyrethroids, for example
   acrinathrin, allethrin (d-cis-trans, d-trans), beta-cyfluthrin, bifenthrin, bioallethrin, bioallethrin-S cyclopentyl isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, deltamethrin, empenthrin (1R isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R trans-isomer), prallethrin, profluthrin, protrifenbute, pyresmethrin, resmethrin, RU 15525, silafluofen, tau-fluvalinate, tefluthrin, terallethrin, tetramethrin (1R isomer), tralomethrin, transfluthrin, ZXI 8901, pyrethrins (pyrethrum)
   DDT
2.2 Oxadiazines, for example indoxacarb
3. Acetylcholine receptor agonists/antagonists
3.1 Chloronicotinyls, for example
   acetamiprid, clothianidin, dinotefuran, imidacloprid, nitenpyram, nithiazine, thiacloprid, thiamethoxam
3.2 Nicotine, bensultap, cartap
4. Acetylcholine receptor modulators
4.1 Spinosyns, for example spinosad
5. GABA-controlled chloride channel antagonists
5.1 Cyclodiene organochlorines, for example
   camphechlor, chlordane, endosulfan, gamma-HCH, HCH, heptachlor, lindane, methoxychlor
5.2 Fiproles, for example
   acetoprole, ethiprole, fipronil, vaniliprole
6. Chloride channel activators
6.1 Mectins, for example
   avermectin, emamectin, emamectin-benzoate, ivemmectin, milbemycin
7. Juvenile hormone mimetics, for example
   diofenolan, epofenonane, fenoxycarb, hydroprene, kinoprene, methoprene, pyriproxifen, triprene
8. Ecdyson agonists/disruptors
8.1 Diacylhydrazines, for example
   chromafenozide, halofenozide, methoxyfenozide, tebufenozide
9. Chitin biosynthesis inhibitors
9.1 Benzoylureas, for example
   bistrifluoron, chlofluazuron, diflubenzuron, fluazuron, flucycloxuron, flufenoxuron, hexaflumuron, lufenuron, novaluron, noviflumuron, penfluoron, teflubenzuron, triflumuron
9.2 Buprofezin
9.3 Cyromazine
10. Oxidative phosphorylation inhibitors, ATP disruptors
10.1 Diafenthiuron
10.2 Organotins, for example azocyclotin, cyhexatin, fenbutatin-oxide
11. Oxidative phosphorylation decouplers acting by interrupting the H-proton gradient
11.1 Pyrroles, for example chlorfenapyr
11.2 Dinitrophenols, for example binapacyrl, dinobuton, dinocap, DNOC
12. Site-I electron transport inhibitors
12.1 METIs, for example fenazaquin, fenpyroximate, pyrimidifen, pyridaben, tebufenpyrad, tolfenpyrad
12.2 Hydramethylnon
12.3 Dicofol
13. Site-II electron transport inhibitors
   Rotenone
14. Site-III electron transport inhibitors
   Acequinocyl, fluacrypyrim
15. Microbial disruptors of the insect gut membrane
   Bacillus thuringiensis strains
16. Fat synthesis inhibitors
   Tetronic acids, for example
      spirodiclofen, spiromesifen
   Tetramic acids, for example
      3-(2,5-dimethylphenyl)-8-methoxy-2-oxo-1-azaspiro[4.5]dec-3-en-4-yl ethyl carbonate (aka: carbonic acid, 3-(2,5-dimethylphenyl)-8-methoxy-2-oxo-1-azaspiro[4.5]dec-3-en-4-yl ethyl ester, CAS-Reg.-No.: 382608-10-8) and carbonic acid, cis-3-(2,5-dimethylphenyl)-8-methoxy-2-oxo-1-azaspiro[4.5]dec-3-en-4-yl ethyl ester (CAS-Reg.-No.: 203313-25-1)
17. Carboxamides, for example flonicamid
18. Octopaminergic agonists, for example amitraz
19. Inhibitors of magnesium-stimulated ATPase, for example propargite
20. BDCAs, for example N-2-[1,1-dimethyl-2-(methylsulphonyl)ethyl]-3-iodo-N1-[2-methyl-4-[1,2,2,2-tetrafluoro-1-(trifluoromethyl)ethyl]phenyl]-1,2-benzenedicarboxamide (CAS-Reg.-No.: 272451-65-7)
21. Nereistoxin analogues, for example thiocyclam hydrogen oxalate, thiosultap-sodium
22. Biologicals, hormones or pheromones, for example
   azadirachtin, Bacillus spec., Beauveria spec., codlemone, Metarrhizium spec., Paecilomyces spec., thuringiensin, Verticillium spec.
23. Active compounds with unknown or unspecific mechanisms of action
23.1 Fumigants, for example
   aluminium phosphide, methyl bromide, sulphuryl fluoride
23.2 Selective antifeedants, for example
   cryolite, flonicamid, pymetrozine
23.3 Mite growth inhibitors, for example
   clofentezine, etoxazole, hexythiazox
23.4 Amidoflumet, benclothiaz, benzoximate, bifenazate, bromopropylate, bupro-fezin, quinomethionate, chlordimeform, chlorobenzilate, chloropicrin, clothiazoben, cycloprene, dicyclanil, fenoxacrim, fentrifanil, flubenzimine, flu-fenerim, flutenzin, gossyplure, hydramethylnone, japonilure, metoxadiazone, petroleum, piperonyl butoxide, potassium oleate, pyridalyl, sulfluramid, tetra-difon, tetrasul, triarathene, verbutin,
   and also products which comprise insecticidal plant extracts, nematodes, fungi or viruses.

A mixture with other known active compounds, such as herbicides, or with fertilizers and growth regulators, safeners and/or semiochemicals is also possible.

When used as insecticides in their commercially available formulations and in the use forms prepared with these formulations, the active compounds according to the invention can furthermore be present in the form of a mixture with synergists. Synergists are compounds by which the activity of the active compounds is increased without it being necessary for the synergist added to be active itself.

When used as insecticides in their commercially available formulations and in the use forms prepared with these formulations, the active compounds according to the invention can furthermore be present in the form of a mixture with inhibitors which reduce the degradation of the active compound after application in the habitat of the plant, on the surface of parts of plants or in plant tissues.

The active compound content of the use forms prepared from the commercially available formulations can vary within broad ranges. The active compound concentration of the use forms can be from 0.0000001 up to 95% by weight of active compound, preferably between 0.0001 and 1% by weight.

They are applied in a customary manner adapted to suit the use forms.

When used against hygiene pests and pests of stored products, the active compound is distinguished by excellent residual action on wood and clay as well as good stability to alkali on limed substrates.

As already mentioned above, it is possible to treat all plants and their parts in accordance with the invention. In a preferred embodiment, wild plant species or plant varieties and plant cultivars which have been obtained by traditional biological breeding methods, such as hybridization or protoplast fusion, and the parts of these varieties and cultivars are treated. In a further preferred embodiment, transgenic plants and plant cultivars which have been obtained by recombinant methods, if appropriate in combination with conventional methods (genetically modified organisms), and their parts are treated. The term “parts” or “parts of plants” or “plant parts” has been explained above.

Plants which are treated particularly preferably in accordance with the invention are those of the plant cultivars which are in each case commercially available or in use. Plant cultivars are understood as meaning plants with new traits which have been bred either by conventional breeding, by mutagenesis or by recombinant DNA techniques. They may take the form of cultivars, biotypes and genotypes.

Depending on the plant species or plant cultivars, their location and growth conditions (soils, climate, vegetation period, nutrition), the treatment according to the invention may also result in superadditive (“synergistic”) effects. Thus, for example, reduced application rates and/or a widened activity spectrum and/or an increase in the activity of the substances and compositions which can be used in accordance with the invention, better plant growth, increased tolerance to high or low temperatures, increased tolerance to drought or to salinity in the water or soil, increased flowering performance, facilitated harvesting, accelerated maturation, higher yields, higher quality and/or better nutritional value of the harvested products, better storage characteristics and/or processability of the harvested products are possible which exceed the effects which were actually to be expected.

The preferred transgenic plants or plant cultivars (those obtained by recombinant methods) to be treated in accordance with the invention include all those plants which, owing to the process of recombinant modification, were given genetic material which confers particular, advantageous, valuable traits to these plants. Examples of such properties are better plant growth, increased tolerance to high or low temperatures, increased tolerance to drought or to salinity in the water or soil, increased flowering performance, facilitated harvesting, accelerated maturation, higher yields, higher quality and/or higher nutritional value of the harvested products, better storage characteristics and/or better processability of the harvested products. Further examples of such traits, examples which must be mentioned especially, are better defense of the plants against animal and microbial pests, such as against insects, mites, phytopathogenic fungi, bacteria and/or viruses and an increased tolerance of the plants to certain herbicidal active compounds. Examples of transgenic plants which may be mentioned are the important crop plants, such as cereals (wheat, rice), maize, soybeans, potato, cotton, tobacco, oilseed rape and fruit plants (with the fruits apples, pears, citrus fruits and grapes), with particular emphasis on maize, soybeans, potatoes, cotton, tobacco, and oilseed rape. Traits which are especially emphasized are the increased defense of the plants against insects, arachnids, nematodes and slugs and snails, owing to toxins being formed in the plants, in particular toxins which are generated in the plants by the genetic material of Bacillus thuringiensis (for example by the genes CryIA(a), CryIA(b), CryIA(c), CrylIA, CryIIIA, CryIIIB2, Cry9c Cry2Ab, Cry3Bb and CryIF and their combinations; hereinbelow “Bt plants”). Other traits which are particularly emphasized are the increased defence of plants against fungi, bacteria and viruses by the systemic acquired resistance (SAR), systemin, phytoalexins, elicitors and resistance genes and correspondingly expressed proteins and toxins. Other traits which are especially emphasized are the increased tolerance of the plants to certain herbicidal active compounds, for example imidazolinones, sulphonylureas, glyphosate or phosphinotricin (for example “PAT” gene). The genes which confer the desired traits in each case may also be present in the transgenic plants in combination with one another. Examples of “Bt plants” which may be mentioned are maize cultivars, cotton cultivars, soybean cultivars and potato cultivars which are commercially available under the trade names YIELD GARD® (for example maize, cotton, soybeans), KnockOut® (for example maize), StarLink® (for example maize), Bollgard® (cotton), Nucotn® (cotton) and NewLeaf® (potato). Examples of herbicide-tolerant plants which may be mentioned are maize cultivars, cotton cultivars and soybean cultivars which are commercially available under the trade names Roundup Ready® (tolerance to glyphosate, for example maize, cotton, soybean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned also include the varieties commercially available under the name Clearfield® (for example maize). Naturally, these statements also apply to plant cultivars having these genetic traits or genetic traits still to be developed, which plant cultivars will be developed and/or marketed in the future.

The plants listed can be treated particularly advantageously according to the invention with the compounds of the general formula I or the active compound mixtures according to the invention. The preferred ranges stated above for the active compounds and mixtures also apply to the treatment of these plants. Particular emphasis may be given to the treatment of plants with the compounds or mixtures specifically mentioned in the present text.

The active compounds according to the invention are not only active against plant, hygiene and stored-product pests, but also, in the veterinary medicine sector, against animal parasites (ectoparasites), such as ixodid ticks, argasid ticks, scab mites, trombi-culid mites, flies (stinging and sucking), parasitic fly larvae, lice, hair lice, bird lice and fleas. These parasites include:

From the order of the Anoplurida, for example, Haematopinus spp., Linognathus spp., Pediculus spp., Phtirus spp., Solenopotes spp.

From the order of the Mallophagida and the sub-orders Amblycerina and Ischnocerina, for example, Trimenopon spp., Menopon spp., Trinoton spp., Bovicola spp., Werneckiella spp., Lepikentron spp., Damalina spp., Trichodectes spp., Felicola spp.

From the order of the Diptera and the sub-orders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

From the order of the Siphonapterida, for example, Pulex spp., Ctenocephalides spp., Xenopyslla spp. and Ceratophyllus spp.

From the order of the Heteropterida, for example, Cimex spp., Triatoma spp., Rhodnius spp. and Panstrongylus spp.

From the order of the Blattarida, for example, Blatta orientalis, Periplaneta americana, Blattela germanica and Supella spp.

From the sub-class of the Acaria (Acarina) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Stemostoma spp. and Varroa spp.

From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The active compounds of the formula (I) according to the invention are also suitable for controlling arthropods which attack agricultural livestock, such as, for example, cattle, sheep, goats, horses, pigs, donkeys, camels, buffaloes, rabbits, chickens, turkeys, ducks, geese, honeybees, other domestic animals, such as, for example, dogs, cats, cage birds, aquarium fish, and so-called experimental animals, such as, for example, hamsters, guinea-pigs, rats and mice. By combating these arthropods, it is intended to reduce deaths and decreased performances (in meat, milk, wool, hides, eggs, honey and the like), so that more economical and simpler animal keeping is made possible by using the active compounds according to the invention.

In the veterinary sector, the active compounds according to the invention are used in a known manner by enteral administration, for example in the form of tablets, capsules, drinks, drenches, granules, pastes, boli, the feed-through method, suppositories, by parenteral administration, such as, for example, by means of injections (intramuscular, subcutaneous, intravenous, intraperitoneal and the like), implants, by nasal application, by dermal administration, for example in the form of dipping or bathing, spraying, pouring-on and spotting-on, washing, dusting, and with the aid of shaped articles which comprise active compound, such as collars, ear tags, tail marks, limb bands, halters, marking devices and the like.

When administered to livestock, poultry, domestic animals and the like, the active compounds of the formula (I) can be used as formulations (for example powders, emulsions, flowables) which comprise the active compounds in an amount of 1 to 80% by weight, either directly or after dilution by a factor of 100 to 10 000, or they may be used in the form of a chemical bath.

Furthermore, it has been found that the compounds according to the invention have a potent insecticidal action against insects which destroy industrial materials.

The following insects may be mentioned by way of example and as being preferred, but without any limitation:

Beetles, such as

Hylotrupes bajulus, Chlorophorus pilosis, Anobium punctatum, Xestobium rufovillosum, Ptilinus pecticomis, Dendrobium pertinex, Emobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec., Tryptodendron spec., Apate monachus, Bostrychus capucins, Heterobostrychus brunneus, Sinoxylon spec., Dinoderus minutus.

Dermapterans, such as

Sirex juvencus, Urocerus gigas, Urocerus gigas taignus, Urocerus augur.

Termites, such as

Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus.

Bristletails, such as Lepisma saccharina.

Industrial materials are to be understood as meaning, in the present context, non-live materials, such as, preferably, synthetic materials, glues, sizes, paper and board, leather, wood and timber products, and paint.

The materials to be very particularly preferably protected against attack by insects are wood and timber products.

Wood and timber products which can be protected by the composition according to the invention or mixtures comprising such a composition are to be understood as meaning, for example:

construction timber, wooden beams, railway sleepers, bridge components, jetties, wooden vehicles, boxes, pallets, containers, telephone poles, wood cladding, windows and doors made of wood, plywood, particle board, joiner's articles, or wood products which, quite generally, are used in the construction of houses or in joinery.

The active compounds can be used as such, in the form of concentrates or generally customary formulations, such as powders, granules, solutions, suspensions, emulsions or pastes.

The formulations mentioned can be prepared in a manner known per se, for example by mixing the active compounds with at least one solvent or diluent, emulsifier, dispersant and/or binder or fixative, water repellent, if appropriate desiccants and UV stabilizers and, if appropriate, colorants and pigments and other processing auxiliaries.

The insecticidal compositions or concentrates used for the protection of wood and wooden materials comprise the active compound according to the invention in a concentration of 0.0001 to 95% by weight, in particular 0.001 to 60% by weight.

The amount of the compositions or concentrates employed depends on the species and the occurrence of the insects and on the medium. The optimum rate of application can be determined upon use in each case by a test series. However, in general, it suffices to employ 0.0001 to 20% by weight, preferably 0.001 to 10% by weight, of the active compound, based on the material to be protected.

The solvent and/or diluent used is an organochemical solvent or solvent mixture and/or an oily or oil-type organochemical solvent or solvent mixture of low volatility and/or a polar organochemical solvent or solvent mixture and/or water and, if appropriate, an emulsifier and/or wetting agent.

Organochemical solvents which are preferably employed are oily or oil-type solvents having an evaporation number of above 35 and a flashpoint of above 30° C., preferably above 45° C. Substances which are used as such oily and oil-type solvents which have low volatility and are insoluble in water are suitable mineral oils or their aromatic fractions, or mineral-oil-containing solvent mixtures, preferably white spirit, petroleum and/or alkylbenzene.

Substances which are advantageously used are mineral oils with a boiling range of 170 to 220° C., white spirit with a boiling range of 170 to 220° C., spindle oil with a boiling range of 250 to 350° C., petroleum or aromatics of boiling range 160 to 280° C., essence of terpentine and the like.

In a preferred embodiment, liquid aliphatic hydrocarbons with a boiling range of 180 to 210° C. or high-boiling mixtures of aromatic and aliphatic hydrocarbons with a boiling range of 180 to 220° C. and/or spindle oil and/or monochloronaphthalene, preferably α-monochloronaphthalene, are used.

The organic oily or oil-type solvents of low volatility having an evaporation number of above 35 and a flashpoint of above 30° C., preferably above 45° C., can be partially replaced by organochemical solvents of high or medium volatility, with the proviso that the solvent mixture also has an evaporation number of above 35 and a flashpoint of above 30° C., preferably above 45° C., and that the insecticide/fungicide mixture is soluble or emulsifiable in this solvent mixture.

In a preferred embodiment, part of the organochemical solvent or solvent mixture or an aliphatic polar organochemical solvent or solvent mixture is replaced. Substances which are preferably used are aliphatic organochemical solvents having hydroxyl and/or ester and/or ether groups, such as, for example, glycol ethers, esters and the like.

The organochemical binders used within the scope of the present invention are the synthetic resins and/or binding drying oils which are known per se and can be diluted with water and/or are soluble or dispersible or emulsifiable in the organochemical solvents employed, in particular binders composed of, or comprising, an acrylate resin, a vinyl resin, for example polyvinyl acetate, polyester resin, polycondensation or polyaddition resin, polyurethane resin, alkyd resin or modified alkyd resin, phenol resin, hydrocarbon resin, such as indene/cumarone resin, silicone resin, drying vegetable and/or drying oils and/or physically drying binders based on a natural and/or synthetic resin.

The synthetic resin used as the binder can be employed in the form of an emulsion, dispersion or solution. Up to 10% by weight of bitumen or bituminous substances can also be used as binders. In addition, colorants, pigments, water repellents, odour-masking substances and inhibitors or anticorrosives known per se and the like can also be employed.

The composition or the concentrate preferably comprises, in accordance with the invention, at least one alkyd resin or modified alkyd resin and/or a drying vegetable oil as the organochemical binder. Preferably used according to the invention are alkyd resins with an oil content of over 45% by weight, preferably 50 to 68% by weight.

All or some of the abovementioned binder can be replaced by a fixative (mixture) or a plasticizer (mixture). These additives are intended to prevent volatilization of the active compounds and crystallization or precipitation. They preferably replace 0.01 to 30% of the binder (based on 100% of binder employed).

The plasticizers are from the chemical classes of the phthalic esters, such as dibutyl phthalate, dioctyl phthalate or benzyl butyl phthalate, the phosphoric esters, such as tributyl phosphate, the adipic esters, such as di-(2-ethylhexyl) adipate, the stearates, such as butyl stearate or amyl stearate, the oleates, such as butyl oleate, the glycerol ethers or relatively high-molecular-weight glycol ethers, glycerol esters and p-toluenesulphonic esters.

Fixatives are chemically based on polyvinyl alkyl ethers, such as, for example, polyvinyl methyl ether, or ketones, such as benzophenone or ethylenebenzophenone.

Particularly suitable as a solvent or diluent is also water, if appropriate as a mixture with one or more of the abovementioned organochemical solvents or diluents, emulsifiers and dispersants.

Particularly effective protection of wood is achieved by large-scale industrial impregnation processes, for example vacuum, double-vacuum or pressure processes.

If appropriate, the ready-to-use compositions can additionally comprise other insecticides and, if appropriate, additionally one or more fungicides.

Suitable additional components which may be admixed are, preferably, the insecticides and fungicides mentioned in WO 94/29 268. The compounds mentioned in that document are expressly part of the present application.

Very particularly preferred components which may be admixed are insecticides, such as chlorpyriphos, phoxim, silafluofin, alphamethrin, cyfluthrin, cypermethrin, deltamethrin, permethrin, imidacloprid, NI-25, flufenoxuron, hexaflumuron, transfluthrin, thiacloprid, methoxyphenoxid and triflumuron, chlothianidin, spinosad, tefluthrin,

and fungicides, such as epoxyconazole, hexaconazole, azaconazole, propiconazole, tebuconazole, cyproconazole, metconazole, imazalil, dichlorfluanid, tolylfluanid, 3-iodo-2-propynylbutyl carbamate, N-octyl-isothiazolin-3-one and 4,5-dichloro-N-octylisothiazolin-3-one.

The compounds according to the invention can at the same time be employed for protecting objects which come into contact with salt water or brackish water, in particular hulls, screens, nets, buildings, moorings and signalling systems, against fouling.

Fouling by sessile Oligochaeta, such as Serpulidae, and by shells and species from the Ledamorpha group (goose barnacles), such as various Lepas and Scalpellum species, or by species from the Balanomorpha group (acorn barnacles), such as Balanus or Pollicipes species, increases the frictional drag of ships and, as a consequence, leads to a marked increase in operation costs owing to higher energy consumption and additionally frequent residence in the dry dock.

Apart from fouling by algae, for example Ectocarpus sp. and Ceramium sp., fouling by sessile Entomostraka groups, which come under the generic term Cirripedia (cirriped crustaceans), is of particular importance.

Surprisingly, it has now been found that the compounds according to the invention, alone or in combination with other active compounds, have an outstanding antifouling action.

Using the compounds according to the invention, alone or in combination with other active compounds, allows the use of heavy metals such as, for example, in bis-(trialkyltin) sulphides, tri-n-butyltin laurate, tri-n-butyltin chloride, copper(I) oxide, triethyltin chloride, tri-n-butyl-(2-phenyl-4-chlorophenoxy)tin, tributyltin oxide, molybdenum disulphide, antimony oxide, polymeric butyl titanate, phenyl-(bispyridine)-bismuth chloride, tri-n-butyltin fluoride, manganese ethylenebisthio-carbamate, zinc dimethyldithiocarbamate, zinc ethylenebisthiocarbamate, zinc salts and copper salts of 2-pyridinethiol 1-oxide, bisdimethyldithiocarbamoylzinc ethylene-bisthiocarbamate, zinc oxide, copper(I) ethylene-bisdithiocarbamate, copper thiocyanate, copper naphthenate and tributyltin halides to be dispensed with, or the concentration of these compounds to be substantially reduced.

If appropriate, the ready-to-use antifouling paints can additionally comprise other active compounds, preferably algicides, fungicides, herbicides, molluscicides, or other antifouling active compounds.

Preferably suitable components in combination with the antifouling compositions according to the invention are:

algicides such as

2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn;

fungicides such as

benzo[b]thiophenecarboxylic acid cyclohexylamide S,S-dioxide, dichlofluanid, fluorfolpet, 3-iodo-2-propinyl butylcarbamate, tolylfluanid and azoles such as

azaconazole, cyproconazole, epoxyconazole, hexaconazole, metconazole, propiconazole and tebuconazole;

molluscicides such as

fentin acetate, metaldehyde, methiocarb, niclosamid, thiodicarb and trimethacarb; Fe chelates;

or conventional antifouling active compounds such as

4,5-dichloro-2-octyl-4-isothiazolin-3-one, diiodomethylparatryl sulphone, 2-(N,N-dimethylthiocarbamoylthio)-5-nitrothiazyl, potassium, copper, sodium and zinc salts of 2-pyridinethiol 1-oxide, pyridine-triphenylborane, tetrabutyldistannoxane, 2,3,5,6-tetrachloro-4-(methylsulphonyl)-pyridine, 2,4,5,6-tetrachloroisophthalonitrile, tetra-methylthiuram disulphide and 2,4,6-trichlorophenylmaleimide.

The antifouling compositions used comprise the active compound according to the invention of the compounds according to the invention in a concentration of 0.001 to 50% by weight, in particular 0.01 to 20% by weight.

Moreover, the antifouling compositions according to the invention comprise the customary components such as, for example, those described in Ungerer, Chem. Ind. 1985, 37, 730-732 and Williams, Antifouling Marine Coatings, Noyes, Park Ridge, 1973.

Besides the algicidal, fungicidal, molluscicidal active compounds and insecticidal active compounds according to the invention, antifouling paints comprise, in particular, binders.

Examples of recognized binders are polyvinyl chloride in a solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants which are preferably insoluble in salt water. Paints may furthermore comprise materials such as rosin to allow controlled release of the active compounds. Furthermore, the paints may comprise plasticizers, modifiers which affect the rheological properties and other conventional constituents. The compounds according to the invention or the abovementioned mixtures may also be incorporated into self-polishing antifouling systems.

The active compounds are also suitable for controlling animal pests, in particular insects, arachnids and mites, which are found in enclosed spaces such as, for example, dwellings, factory halls, offices, vehicle cabins and the like. They can be employed in domestic insecticide products for controlling these pests alone or in combination with other active compounds and auxiliaries. They are active against sensitive and resistant species and against all development stages. These pests include:

From the order of the Scorpionidea, for example, Buthus occitanus.

From the order of the Acarina, for example, Argas persicus, Argas reflexus, Bryobia ssp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.

From the order of the Araneae, for example, Aviculariidae, Araneidae.

From the order of the Opiliones, for example, Pseudoscorpiones chelifer, Pseudoscorpiones cheiridium, Opiliones phalangium.

From the order of the Isopoda, for example, Oniscus asellus, Porcellio scaber.

From the order of the Diplopoda, for example, Blaniulus guttulatus, Polydesmus spp.

From the order of the Chilopoda, for example, Geophilus spp.

From the order of the Zygentoma, for example, Ctenolepisma spp., Lepisma saccharina, Lepismodes inquilinus.

From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example, Acheta domesticus.

From the order of the Dermaptera, for example, Forficula auricularia.

From the order of the Isoptera, for example, Kaloternes spp., Reticulitermes spp.

From the order of the Psocoptera, for example, Lepinatus spp., Liposcelis spp.

From the order of the Coleptera, for example, Anthrenus spp., Attagenus spp., Dernestes spp., Latheticus oryzae, Necrobia spp., Ptinus spp., Rhizopertha dominica, Sitophilus granarius, Sitophilus oryzae, Sitophilus zeamais, Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa.

From the order of the Lepidoptera, for example, Achroia grisella, Galleria mellonella, Plodia interpunctella, Tinea cloacella, Tinea pellionella, Tineola bisselliella.

From the order of the Siphonaptera, for example, Ctenocephalides canis, Ctenocephalides felis, Pulex irritans, Tunga penetrans, Xenopsylla cheopis.

From the order of the Hymenoptera, for example, Camponotus herculeanus, Lasius fuliginosus, Lasius niger, Lasius umbratus, Monomorium pharaonis, Paravespula spp., Tetramorium caespitum.

From the order of the Anoplura, for example, Pediculus humanus capitis, Pediculus humanus corporis, Phthirus pubis.

From the order of the Heteroptera, for example, Cimex hemipterus, Cimex lectularius, Rhodinus prolixus, Triatoma infestans.

They are used in the household insecticides sector alone or in combination with other suitable active compounds such as phosphoric esters, carbamates, pyrethroids, neonicotinoids, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

The active compounds according to the invention can also be used as defoliants, desiccants, haulm killers and, in particular, as weed killers. Weeds in the broadest sense are understood as meaning all plants which grow at locations where they are undesired. Whether the substances according to the invention act as nonselective or selective herbicides depends essentially on the application rate.

The active compounds according to the invention can be used, for example, in the following plants:

Dicotyledonous weeds of the genera: Abutilon, Amaranthus, Ambrosia, Anoda, Anthemis, Aphanes, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindemia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

Dicotyledonous crops of the genera: Arachis, Beta, Brassica, Cucumis, Cucurbita, Helianthus, Daucus, Glycine, Gossypium, Ipomoea, Lactuca, Linum, Lycopersicon, Nicotiana, Phaseolus, Pisum, Solanum, Vicia.

Monocotyledonous weeds of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Monocotyledonous crops of the genera: Allium, Ananas, Asparagus, Avena, Hordeum, Oryza, Panicum, Saccharum, Secale, Sorghum, Triticale, Triticum, Zea.

However, the use of the active compounds according to the invention is in no way restricted to these genera, but extends in the same manner to other plants.

Depending on the concentration, the active compounds according to the invention are suitable for the nonselective weed control on, for example, industrial terrains and railway tracks and on paths and locations with and without trees. Likewise the active compounds according to the invention can be employed for controlling weeds in perennial crops, for example forests, ornamental tree plantings, orchards, vineyards, citrus groves, nut orchards, banana plantations, coffee plantations, tea plantations, rubber plantations, oil palm plantations, cocoa plantations, soft fruit plantings and hop fields, on lawns, turf and pastureland, and for the selective control of weeds in annual crops.

The compounds of the formula (I) according to the invention have strong herbicidal activity and a broad activity spectrum when used on the soil and on aerial plant parts. To a certain extent, they are also suitable for the selective control of monocotyledonous and dicotyledonous weeds in monocotyledonous and dicotyledonous crops, both pre- and post-emergence.

At certain concentrations or application rates, the active compounds according to the invention can also be employed for controlling animal pests and fungal or bacterial plant diseases. If appropriate, they can also be used as intermediates or precursors for the synthesis of other active compounds.

The active compounds can be converted into the customary formulations, such as solutions, emulsions, wettable powders, suspensions, powders, dusting agents, pastes, soluble powders, granules, suspoemulsion concentrates, natural and synthetic materials impregnated with active compound, and very fine capsules in polymeric substances.

These formulations are produced in a known manner, for example by mixing the active compounds with extenders, that is liquid solvents and/or solid carriers, optionally with the use of surfactants, that is emulsifiers and/or dispersants and/or foam-formers.

If the extender used is water, it is also possible to use, for example, organic solvents as auxiliary solvents. Suitable liquid solvents are essentially: aromatics, such as xylene, toluene or alkylnaphthalenes, chlorinated aromatics and chlorinated aliphatic hydrocarbons, such as chlorobenzenes, chloroethylenes or methylene chloride, aliphatic hydrocarbons, such as cyclohexane or paraffins, for example petroleum fractions, mineral and vegetable oils, alcohols, such as butanol or glycol, and also their ethers and esters, ketones, such as acetone, methyl ethyl ketone, methyl isobutyl ketone or cyclohexanone, strongly polar solvents, such as dimethylformamide and dimethyl sulphoxide, and also water.

Suitable solid carriers are: for example ammonium salts and ground natural minerals, such as kaolins, clays, talc, chalk, quartz, attapulgite, montmorillonite or diatomaceous earth, and ground synthetic minerals, such as finely divided silica, alumina and silicates, suitable solid carriers for granules are: for example crushed and fractionated natural rocks such as calcite, marble, pumice, sepiolite and dolomite, and also synthetic granules of inorganic and organic meals, and granules of organic material such as sawdust, coconut shells, maize cobs and tobacco stalks; suitable emulsifiers and/or foam-formers are: for example non-ionic and anionic emulsifiers, such as poly-oxyethylene fatty acid esters, polyoxyethylene fatty alcohol ethers, for example alkylaryl polyglycol ethers, alkylsulphonates, alkyl sulphates, arylsulphonates and protein hydrolysates; suitable dispersants are: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, and also natural phospholipids, such as cephalins and lecithins, and synthetic phospholipids, can be used in the formulations. Other possible additives are mineral and vegetable oils.

It is possible to use colorants such as inorganic pigments, for example iron oxide, titanium oxide and Prussian Blue, and organic colorants, such as alizarin colorants, azo colorants and metal phthalocyanine colorants, and trace nutrients such as salts of iron, manganese, boron, copper, cobalt, molybdenum and zinc.

The formulations generally comprise between 0.1 and 95 percent by weight of active compound, preferably between 0.5 and 90%.

The active compounds according to the invention, as such or in their formulations, can also be used for weed control purposes as a mixture with known herbicides and/or with substances which improve crop plant tolerance (“safeners”), ready mixes or tank mixes being possible. Mixtures with herbicide products which contain one or more known herbicides and a safener are hence also possible.

Herbicides which are suitable for the mixtures are known herbicides, for example

acetochlor, acifluorfen (-sodium), aclonifen, alachlor, alloxydim (-sodium), ametryne, amicarbazone, amidochlor, amidosulfuron, anilofos, asulam, atrazine, aza-fenidin, azimsulfuron, beflubutamid, benazolin (-ethyl), benfuresate, bensulfuron (-methyl), bentazone, benzfendizone, benzobicyclon, benzofenap, benzoylprop (-ethyl), bialaphos, bifenox, bispyribac (-sodium), bromobutide, bromofenoxim, bromoxynil, butachlor, butafenacil (-allyl), butroxydim, butylate, cafenstrole, caloxy-dim, carbetamide, carfentrazone (-ethyl), chlomethoxyfen, chloramben, chloridazon, chlorimuron (-ethyl), chlornitrofen, chlorsulfuron, chlortoluron, cinidon (-ethyl), cin-methylin, cinosulfuron, clefoxydim, clethodim, clodinafop (-propargyl), clomazone, clomeprop, clopyralid, clopyrasulfuron (-methyl), cloransulam (-methyl), cumyluron, cyanazine, cybutryne, cycloate, cyclosulfamuron, cycloxydim, cyhalofop (-butyl), 2,4-D, 2,4-DB, desmedipham, diallate, dicamba, dichlorprop (—P), diclofop (-methyl), diclosulam, diethatyl (-ethyl), difenzoquat, diflufenican, diflufenzopyr, dimefuron, dimepiperate, dimethachlor, dimethametryn, dimethenamid, dimexyflam, di-nitramine, diphenamid, diquat, dithiopyr, diuron, dymron, epropodan, EPTC, espro-carb, ethalfluralin, ethametsulfuron (-methyl), ethofumesate, ethoxyfen, ethoxysulf-uron, etobenzanid, fenoxaprop (—P-ethyl), fentrazamide, flamprop (-isopropyl, -iso-propyl-L, -methyl), flazasulfuron, florasulam, fluazifop (—P-butyl), fluazolate, flu-carbazone (-sodium), flufenacet, flumetsulam, flumiclorac (-pentyl), flumioxazin, flumipropyn, flumetsulam, fluometuron, fluorochloridone, fluoroglycofen (-ethyl), flupoxam, flupropacil, flurpyrsulfuron (-methyl, -sodium), flurenol (-butyl), fluridone, fluoroxypyr (-butoxypropyl, -meptyl), flurprimidol, flurtamone, fluthiacet (-methyl), fluthiamide, fomesafen, foramsulfuron, glufosinate (-ammonium), gly-phosate (-isopropylammonium), halosafen, haloxyfop (-ethoxyethyl, —P-methyl), hex-azinone, imazamethabenz (-methyl), imazamethapyr, imazamox, imazapic, imazapyr, imazaquin, imazethapyr, imazosulfuron, iodosulfuron (-methyl, -sodium), ioxynil, isopropalin, isoproturon, isouron, isoxaben, isoxachlortole, isoxaflutole, isoxapyri-fop, lactofen, lenacil, linuron, MCPA, mecoprop, mefenacet, mesotrione, meta-mitron, metazachlor, methabenzthiazuron, metobenzuron, metobromuron, (alpha-) metolachlor, metosulam, metoxuron, metribuzin, metsulfuron (-methyl), molinate, monolinuron, naproanilide, napropamide, neburon, nicosulfuron, norflurazon, orben-carb, oryzalin, oxadiargyl, oxadiazon, oxasulfuron, oxaziclomefone, oxyfluorfen, paraquat, pelargonic acid, pendimethalin, pendralin, pentoxazone, phenmedipham, picolinafen, piperophos, pretilachlor, primisulfuron (-methyl), profluazol, prometryn, propachlor, propanil, propaquizafop, propisochlor, propoxycarbazone (-sodium), propyzamide, prosulfocarb, prosulfuron, pyraflufen (-ethyl), pyrazogyl, pyrazolate, pyrazosulfuron (-ethyl), pyrazoxyfen, pyribenzoxim, pyributicarb, pyridate, pyridatol, pyriftalide, pyriminobac (-methyl), pyrithiobac (-sodium), quinchlorac, quinmerac, quinoclamine, quizalofop (—P-ethyl, —P-tefuryl), rimsulfuron, sethoxydim, simazine, simetryn, sulcotrione, sulfentrazone, sulfometuron (-methyl), sulfosate, sulfosulfuron, tebutam, tebuthiuron, tepraloxydim, terbuthylazine, terbutryn, thenylchlor, thiaflu-amide, thiazopyr, thidiazimin, thifensulfuron (-methyl), thiobencarb, tiocarbazil, tralkoxydim, triallate, triasulfuron, tribenuron (-methyl), triclopyr, tridiphane, tri-fluralin, trifloxysulfuron, triflusulfuron (-methyl), tritosulfuron.

Also suitable for the mixtures are known safeners, for example:

AD-67, BAS-145138, benoxacor, cloquintocet (-mexyl), cyometrinil, 2,4-D, DKA-24, dichlormid, dymmon, fenclorim, fenchlorazol (-ethyl), flurazole, fluxofenim, furilazole, isoxadifen (-ethyl), MCPA, mecoprop (—P), mefenpyr (-diethyl), MG-191, oxabetrinil, PPG-1292, R-29148.

A mixture with other known active compounds, such as fungicides, insectides, acaricides, nematicides, bird repellents, plant nutrients and soil conditioners, is also possible.

The active compounds can be applied as such, in the form of their formulations or the use forms prepared therefrom by further dilution, such as ready-to-use solutions, suspensions, emulsions, powders, pastes and granules. They are applied in the customary manner, for example by pouring, spraying, atomizing, spreading.

The active compounds according to the invention can be applied both before and after plant emergence. They can also be incorporated into the soil prior to planting.

The application rate of active compound can vary within a substantial range. Essentially, it depends on the nature of the desired effect. In general, the application rates are between 1 g and 10 kg of active compound per hectare of soil area, preferably between 5 g and 5 kg per ha.

The substances according to the invention have potent microbicidal activity and can be employed for controlling unwanted microorganisms, such as fungi and bacteria, in crop protection and in the protection of materials.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Some pathogens causing fungal and bacterial diseases which come under the generic names listed above may be mentioned as examples, but not by way of limitation:

Xanthomonas species, such as, for example, Xanthomonas campestris pv. oryzae;

Pseudomonas species, such as, for example, Pseudomonas syringae pv. lachrymans;

Erwinia species, such as, for example, Erwinia amylovora;

Pythium species, such as, for example, Pythium ultimum;

Phytophthora species, such as, for example, Phytophthora infestans;

Pseudoperonospora species, such as, for example, Pseudoperonospora humuli or

Pseudoperonospora cubensis;

Plasmopara species, such as, for example, Plasmopara viticola;

Bremia species, such as, for example, Bremia lactucae;

Peronospora species, such as, for example, Peronospora pisi or P. brassicae;

Erysiphe species, such as, for example, Erysiphe graminis;

Sphaerotheca species, such as, for example, Sphaerotheca fuliginea;

Podosphaera species, such as, for example, Podosphaera leucotricha;

Venturia species, such as, for example, Venturia inaequalis;

Pyrenophora species, such as, for example, Pyrenophora teres or P. graminea (conidia form: Drechslera, syn: Helminthosporium);

Cochliobolus species, such as, for example, Cochliobolus sativus (conidia form: Drechslera, syn: Helminthosporium);

Uromyces species, such as, for example, Uromyces appendiculatus;

Puccinia species, such as, for example, Puccinia recondita;

Sclerotinia species, such as, for example, Sclerotinia sclerotiorum;

Tilletia species, such as, for example, Tilletia caries;

Ustilago species, such as, for example, Ustilago nuda or Ustilago avenae;

Pellicularia species, such as, for example, Pellicularia sasakii;

Pyricularia species, such as, for example, Pyricularia oryzae;

Fusarium species, such as, for example, Fusarium culmorum;

Botrytis species, such as, for example, Botrytis cinerea;

Septoria species, such as, for example, Septoria nodorum;

Leptosphaeria species, such as, for example, Leptosphaeria nodorum;

Cercospora species, such as, for example, Cercospora canescens;

Alternaria species, such as, for example, Alternaria brassicae; and

Pseudocercosporella species, such as, for example, Pseudocercosporella herpotrichoides.

The active compounds according to the invention also have very good fortifying action in plants. Accordingly, they can be used for mobilizing the defenses of the plant against attack by unwanted microorganisms.

In the present context, plant-fortifying (resistance-inducing) substances are to be understood as meaning those substances which are capable of stimulating the defense system of plants such that, when the treated plants are subsequently inoculated with unwanted microorganisms, they show substantial resistance against these microorganisms.

In the present case, unwanted microorganisms are to be understood as meaning phytopathogenic fungi, bacteria and viruses. Accordingly, the substances according to the invention can be used to protect plants for a certain period after the treatment against attack by the pathogens mentioned. The period for which protection is provided generally extends over 1 to 10 days, preferably 1 to 7 days, after the treatment of the plants with the active compounds.

The fact that the active compounds are well tolerated by plants at the concentrations required for controlling plant diseases permits the treatment of above-ground parts of plants, of propagation stock and seeds, and of the soil.

The active compounds according to the invention are also suitable for increasing the yield of crops. In addition, they show reduced toxicity and are well tolerated by plants.

At certain concentrations and application rates, the active compounds according to the invention can if appropriate also be used as herbicides, for influencing plant growth and for controlling animal pests. If appropriate, they can also be used as intermediates and precursors for the synthesis of further active compounds.

In the protection of materials, the compounds according to the invention can be employed for protecting industrial materials against infection with, and destruction by, unwanted microorganisms.

Industrial materials in the present context are understood as meaning non-living materials which have been prepared for use in industry. For example, industrial materials which are intended to be protected by active compounds according to the invention from microbial change or destruction can be adhesives, sizes, paper and board, textiles, leather, wood, paints and plastic articles, cooling lubricants and other materials which can be infected with, or destroyed by, microorganisms. Parts of production plants, for example cooling-water circuits, which may be impaired by the proliferation of microorganisms may also be mentioned within the scope of the materials to be protected. Industrial materials which may be mentioned within the scope of the present invention are preferably adhesives, sizes, paper and board, leather, wood, paints, cooling lubricants and heat-transfer liquids, particularly preferably wood.

Microorganisms capable of degrading or changing the industrial materials which may be mentioned are, for example, bacteria, fungi, yeasts, algae and slime organisms. The active compounds according to the invention preferably act against fungi, in particular moulds, wood-discolouring and wood-destroying fungi (Basidiomycetes), and against slime organisms and algae.

Microorganisms of the following genera may be mentioned as examples:

Alternaria, such as Alternaria tenuis,

Aspergillus, such as Aspergillus niger,

Chaetomium, such as Chaetomium globosum,

Coniophora, such as Coniophora puetana,

Lentinus, such as Lentinus tigrinus,

Penicillium, such as Penicillium glaucum,

Polyporus, such as Polyporus versicolor,

Aureobasidium, such as Aureobasidium pullulans,

Sclerophoma, such as Sclerophoma pityophila,

Trichoderma, such as Trichoderma viride,

Escherichia, such as Escherichia coli,

Pseudomonas, such as Pseudomonas aeruginosa, and

Staphylococcus, such as Staphylococcus aureus.

Depending on their particular physical and/or chemical properties, the active compounds can be converted into the customary formulations, such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the active compounds with extenders, that is liquid solvents, liquefied gases under pressure, and/or solid carriers, optionally with the use of surfactants, that is emulsifiers and/or dispersants and/or foam formers. If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as finely divided silica, alumina and silicates. Suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates. Suitable dispersants are: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic colorants such as alizarin colorants, azo colorants and metal phthalocyanine colorants, and trace nutrients such as salts of iron, manganese, boron, copper, cobalt, molybdenum and zinc.

The formulations generally comprise between 0.1 and 95% by weight of active compound, preferably between 0.5 and 90%.

The active compounds according to the invention can be used as such or in their formulations, also in a mixture with known fungicides, bactericides, acaricides, nematicides or insecticides, to broaden, for example, the activity spectrum or to prevent development of resistance. In many cases, synergistic effects are obtained, i.e. the activity of the mixture is greater than the activity of the individual components.

Suitable mixing components are those specified above

A mixture with other known active compounds, such as herbicides, or with fertilizers and growth regulators, is also possible.

In addition, the compounds of the formula (I) according to the invention also have very good antimycotic activity. They have a very broad antimycotic activity spectrum in particular against dermatophytes and yeasts, moulds and diphasic fungi (for example against Candida species, such as Candida albicans, Candida glabrata), and Epidermophyton floccosum, Aspergillus species, such as Aspergillus niger and Aspergillus fumigatus, Trichophyton species, such as Trichophyton mentagrophytes,

Microsporon species such as Microsporon canis and audouinii. The listing of these fungi by no means limits the mycotic spectrum that can be covered, but is only for illustration.

The active compounds can be used as such, in the form of their formulations or the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, spreading, etc. It is further possible to apply the active compounds by the ultra-low-volume method, or to inject the active compound preparation or the active compound itself into the soil. It is also possible to treat the seeds of the plants.

When using the active compounds according to the invention as fungicides, the application rates can be varied within a relatively wide range, depending on the kind of application. For the treatment of parts of plants, the active compound application rates are generally between 0.1 and 10 000 g/ha, preferably between 10 and 1000 g/ha. For seed dressing, the active compound application rates are generally between 0.001 and 50 g per kilogram of seed, preferably between 0.01 and 10 g per kilogram of seed. For the treatment of the soil, the active compound application rates are generally between 0.1 and 10 000 g/ha, preferably between 1 and 5000 g/ha.

The preparation and the use of the active compounds according to the invention are illustrated by the examples below.

### PREPARATION EXAMPLES

### Example I-1-1

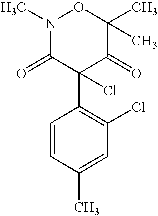

1.49 g (5 mmol) of 4-(2-chloro-4-methylphenyl)-2,6,6-trimethyloxazine-3,5-dione in 5 ml of anhydrous methylene chloride are introduced into a vessel at 0° C. 0.675 g (5 mmol) of sulphuryl chloride is added dropwise. The experiment is stirred overnight at room temperature. It is washed with NaHCO₃ solution, the organic phase is dried and the solvent is removed by distillation.

Yield: 1.37 g (87% of theory), m.p. 178-179° C.

In analogy to Example (I-1-1) and in accordance with the general instructions for preparing compounds of the formula (I-1), the following compounds of the formula (I-1) are obtained

| | |
|---|---|
| | (I-1) |
| 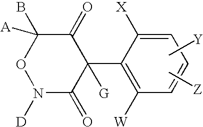 | |

| Ex. | | | | | | | | | |
|---|---|---|---|---|---|---|---|---|---|
| No. | W | X | Y | Z | A | B | D | G | m.p. ° C. |
| 1-1-2 | H | CH₃ | 4-Cl | 5-CH₃ | CH₃ | CH₃ | CH₃ | Cl | 135 |
| 1-1-3 | H | CH₃ | H | 5-(4-Cl—C₆H₄) | —(CH₂)₃— | | CH₃ | Cl | 80-90 |
| 1-1-4 | H | Cl | H | 5-(4-Cl—C₆H₄) | —(CH₂)₃— | | CH₃ | Cl | 131 |
| 1-1-5 | H | CH₃ | H | 5-(4-Cl—C₆H₄) | CH₃ | CH₃ | CH₃ | Cl | 118 |
| 1-1-6 | Cl | CH₃ | 4-Cl | H | CH₃ | CH₃ | CH₃ | Cl | 178-179 |
| 1-1-7 | Cl | C₂H₅ | 4-Cl | H | CH₃ | CH₃ | CH₃ | Cl | 136-137 |
| 1-1-8 | C₂H₅ | C₂H₅ | 4- | H | CH₃ | CH₃ | CH₃ | Cl | 177-178 |
| | | | Br | | | | | | |
| 1-1-9 | CH₃ | Br | 4-Cl | H | CH₃ | CH₃ | CH₃ | Cl | 180-181 |
| 1-1-10 | H | C₂H₅ | 4- | H | CH₃ | CH₃ | CH₃ | Cl | 91-92 |
| | | | Br | | | | | | |
| I-1-11 | H | Cl | H | 5-(4-Cl—C₆H₄)— | CH₃ | CH₃ | CH₃ | Cl | log P 4.58* |
| 1-1-12 | H | CH₃ | H | 5-(4-CF₃—C₆H₄)— | CH₃ | CH₃ | CH₃ | Cl | log P 4.56* |

*The logP values reported in the above tables and preparation examples are determined in accordance with EEC Directive 79/831 Annex V.A8 by means of HPLC (high performance liquid chromatography) on a reversed-phase column (C18). Temperature: 43° C.

The determination is made in the acidic range at a pH of 2.3 using 0.1% aqueous phosphoric acid and acetonitrile as eluents; linear gradient from 10% acetonitrile to 95% acetonitrile.

The determination with LC-MS in the acidic range is made at a pH of 2.7 with 0.1% aqueous formic acid and acetonitrile (containing 0.1% formic acid) as eluents; linear gradient from 10% acetonitrile to 95% acetonitrile.

The determination with LC-MS in the neutral range is made at a pH of 7.8 with 0.001 molar aqueous ammonium hydrogen carbonate solution and acetonitrile as eluents; linear gradient from 10% acetonitrile to 95% acetonitrile.

Calibration is carried out with unbranched alkan-2-ones (having 3 to 16 carbon atoms), for which the logP values are known (logP values determined on the basis of the retention times by means of linear interpolation between two successive alkanones).

The lambda-max values were determined on the basis of the UV spectra from 200 nm to 400 nm in the maxima of the chromatographic signals.

### Example I-2-1

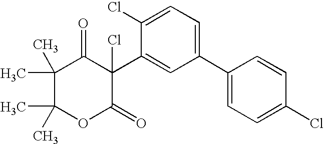

0.15 g of the compound from Ex. I-a-31 (WO 01/98288) in 5 ml of anhydrous chloroform are introduced into a vessel. 0.052 g of sulphuryl chloride in 0.3 ml of anhydrous chloroform is added dropwise with ice cooling. The mixture is stirred for 20 minutes. The reaction solution is washed with 10 ml of 10% strength NaHCO₃ solution and dried and the solvent is removed by distillation.

The residue is then stirred together with cyclohexane/ethyl acetate.

Yield: 0.15 g (92% of theory), m.p. 149° C.

In analogy to Example (I-2-1) and in accordance with the general instructions for preparing compounds of the formula (I-2), the following compounds of the formula

| | |
|---|---|
| | (I-2) |
| 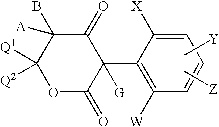 | |

| Ex. | | | | | | | | | | |
|---|---|---|---|---|---|---|---|---|---|---|
| No. | W | X | Y | Z | B | A | Q¹ | Q² | G | m.p. ° C. |
| 1-2-2 | H | CH₃ | H | 5-(4-Cl—C₆H₄) | CH₃ | —(CH₂)₄— | | H | Cl | wax |
| 1-2-3 | H | Cl | H | 5-(4-CF₃—C₆H₄) | CH₃ | CH₃ | CH₃ | CH₃ | Cl | *7.71 (AA′, BB′, 4H, |
| | | | | | | | | | | ArH) 8.16 (d, 1H, |
| | | | | | | | | | | ArH) |
| 1-2-4 | H | Cl | H | 5-(4-F—-C₆H₄) | CH₃ | CH₃ | CH₃ | CH₃ | Cl | *7.15 (“t”, |
| | | | | | | | | | | 2H, ArH) |
| | | | | | | | | | | 8.09 (d, 1H, ArH) |
| 1-2-5 | H | Cl | H | 5-(2,4-F₂—C₆H₃) | CH₃ | CH₃ | CH₃ | CH₃ | Cl | *1.3, 1.44, 1.53, |
| | | | | | | | | | | 1.69 |
| | | | | | | | | | | (4s, 12H, 4CH₃) |
| | | | | | | | | | | 8.03 (s, br, 1H, |
| | | | | | | | | | | ArH) |
| 1-2-6 | H | Cl | H | 5-(2,4-Cl₂—C₆H₃) | CH₃ | CH₃ | CH₃ | CH₃ | Cl | *1.03 (s, 3H, CH₃) |
| | | | | | | | | | | 7.46 (d, 1H, ArH) |
| 1-2-7 | H | CH₃ | H | 5-(4-F—C₆H₄) | CH₃ | CH₃ | CH₃ | CH₃ | Cl | *2.62(s, 3H, Ar, |
| | | | | | | | | | | CH₃) |
| | | | | | | | | | | 7.11 (“t”, 2H, ArH) |
| 1-2-8 | H | CH₃ | H | 5-(4-CF₃—C₆H₄) | CH₃ | CH₃ | CH₃ | CH₃ | Cl | *1.06 (s, 3H, CH₃) |
| | | | | | | | | | | 2.65 (s, 3H, ArH) |
| 1-2-9 | H | CH₃ | H | 5-(4-CF₃—C₆H₄) | CH₃ | CH₃ | CH₃ | CH₃ | Cl | *1.01; 1.02 (2s, 3H, |
| | | | | | | | | | | CH₃) 1.32, 1.34 |
| | | | | | | | | | | (2s, 3H, CH₃) |
| 1-2-10 | H | CH₃ | H | 5-(4-Cl—C₆H₄) | CH₃ | CH₃ | CH₃ | CH₃ | Cl | 182-183 |
| 1-2-11 | H | CH₃ | 4- | 5-(4-Cl—C₆H₄) | CH₃ | CH₃ | CH₃ | CH₃ | Cl | **0.96, 1.24, 1.34, |
| | | | CH₃ | | | | | | | 1.36 (4s, 12H, |
| | | | | | | | | | | 4 × CH₃) |
| 1-2-12 | H | CH₃ | H | 5-(4-Cl—C₆H₄) | —(CH₂)₂—O—(CH₂)₂— | | CH₃ | CH₃ | Cl | **2.12 (s, 3H, |
| | | | | | | | | | | Ar—CH₃) |
| | | | | | | | | | | 3.57-3.63 (m, 2H, |
| | | | | | | | | | | O—CH₂) |
| | | | | | | | | | | 3.73-3.81 (m, 2H, |
| | | | | | | | | | | O—CH₂) |
| 1-2-13 | H | CH₃ | H | 5-(4-Cl—C₆H₄) | CH₃ | CH₃ | H | H | Cl | **1.25, 1.30 (2s, |
| | | | | | | | | | | 6H, 2 × CH₃), |
| | | | | | | | | | | 4.29, 4.7 (2d, |
| | | | | | | | | | | 2H, OCH₂) |
| 1-2-14 | H | Cl | H | 5-(4-F—C₆H₄) | CH₃ | CH₃ | H | H | Cl | **1.33, 1.43 |
| | | | | | | | | | | (2s, 6H, |
| | | | | | | | | | | 2 × CH₃) 4.60, 5.00 |
| | | | | | | | | | | (2d, 2H, CH₂O) |

| | | | | | | | | | |
|---|---|---|---|---|---|---|---|---|---|
| 1-2-15 | H | CH₃ | H | 5-(4-Cl—C₆H₄) | —(CH₂)₃— | H | H | Cl | 162 |

*¹H-NMR (400 MHz, CDCl₃): shifts δ in ppm
**¹H-NMR (400 MHz, d₆-DMSO): shifts δ in ppm

### USE EXAMPLES

### Example No. 1

Phaedon test (PHAECO spray treatment)

| | | |
|---|---|---|
| Solvent: | 78 | parts by weight of acetone |
| | 1.5 | parts by weight of dimethylformamide |
| Emulsifier: | 0.5 | part by weight of alkylaryl polyglycol ether |

A suitable preparation of active compound is prepared by mixing 1 part by weight of active compound with the stated amounts of solvent and emulsifier and diluting the concentrate with emulsifier-containing water to the desired concentration.

Leaf discs of Chinese cabbage (Brassica pekinensis) are sprayed with a preparation of active compound at the desired concentration and after they have dried are populated with larvae of the mustard beetle (Phaedon cochleariae).

After the desired time the effect in % is determined. 100% means that all of the beetle larvae have been killed; 0% means that no beetle larvae have been killed.

In this test the following compounds, for example, of the preparation examples show an activity of 500 g/ha of ≧80%: I-1-3, I-1-4, I-1-5, I-1-10, I-1-11, I-1-12, I-2-3, I-2-4, I-2-5, I-2-10, I-2-12, I-2-13, I-2-14, I-2-15.

### Example No. 2

Spodoptera frugiperda test (SPODFR spray treatment)

| | | |
|---|---|---|
| Solvent: | 78 | parts by weight of acetone |
| | 1.5 | parts by weight of dimethylformamide |
| Emulsifier: | 0.5 | part by weight of alkylaryl polyglycol ether |

A suitable preparation of active compound is prepared by mixing 1 part by weight of active compound with the stated amounts of solvent and emulsifier and diluting the concentrate with emulsifier-containing water to the desired concentration.

Leaf discs of maize (Zea mays) are sprayed with a preparation of active compound at the desired concentration and after they have dried are populated with caterpillars of the army worm (Spodoptera frugiperda).

After the desired time the effect in % is determined. 100% means that all of the caterpillars have been killed; 0% means that no caterpillar has been killed.

In this test the following compounds, for example, of the preparation examples show an activity of 500 g/ha of ≧80%: I-1-3, I-1-4, I-1-5, I-1-10, I-1-11, I-2-3, I-2-4, I-2-5, I-2-6, I-2-10, I-2-12, I-2-13, I-2-14, I-2-15.

### Example No. 3

Myzus test (MYZUPE spray treatment)

| | | |
|---|---|---|
| Solvent: | 78 | parts by weight of acetone |
| | 1.5 | parts by weight of dimethylformamide |
| Emulsifier: | 0.5 | part by weight of alkylaryl polyglycol ether |

A suitable preparation of active compound is prepared by mixing 1 part by weight of active compound with the stated amounts of solvent and emulsifier and diluting the concentrate with emulsifier-containing water to the desired concentration.

Leaf discs of Chinese cabbage (Brassica pekinensis) infested by all stages of the green peach aphid (Myzus persicae) are sprayed with a preparation of active compound at the desired concentration.

After the desired time the effect in % is determined. 100% means that all of the aphids have been killed; 0% means that no aphids have been killed.

In this test the following compounds, for example, of the preparation examples show an activity of 500 g/ha of ≧90%: I-1-10, I-1-12, I-2-2, I-2-3, I-2-4, I-2-5, I-2-12, I-2-13, I-2-14, I-2-15.

### Example No. 4

Tetranychus test, OP-resistent (TETRUR spray treatment)

| | | |
|---|---|---|
| Solvent: | 78 | parts by weight of acetone |
| | 1.5 | parts by weight of dimethylformamide |
| Emulsifier: | 0.5 | part by weight of alkylaryl polyglycol ether |

A suitable preparation of active compound is prepared by mixing 1 part by weight of active compound with the stated amounts of solvent and emulsifier and diluting the concentrate with emulsifier-containing water to the desired concentration.

Leaf discs of bean (Phaseolus vulgaris) infested by all stages of the greenhouse red spider mite (Tetranychus urticae) are sprayed with a preparation of active compound at the desired concentration.

After the desired time the effect in % is determined. 100% means that all of the spider mites have been killed; 0% means that no spider mites have been killed.

In this test the following compounds, for example, of the preparation examples show an activity of 100 g/ha of ≧80%: I-1-3, I-1-4, I-1-12, I-2-12, I-2-13, I-2-15.

### Example No. 5

Phaedon cochleariae - larvae test (PHAECO)

| | | |
|---|---|---|
| Solvent: | 7 | parts by weight of dimethylformamide |
| Emulsifier: | 2 | parts by weight of alkylaryl polyglycol ether |

A suitable preparation of active compound is prepared by mixing 1 part by weight of active compound with the stated amounts of solvent and emulsifier and diluting the concentrate with emulsifier-containing water to the desired concentration.

Leaves of cabbage (Brassica oleracea) are treated by being immersed in the preparation of active compound at the desired concentration and while still wet are populated with larvae of the mustard beetle (Phaedon cochleariae).

After the desired time, the kill rate in % is determined. 100% means that all of the beetle larvae have been killed; 0% means that no beetle larvae have been killed.

In this test the following compounds, for example, of the preparation examples show an activity of 500 ppm of ≧80%: I-2-1, I-2-2.

### Example No. 6

Spodoptera frugiperda test (SPODFR)

| | | |
|---|---|---|
| Solvent: | 7 | parts by weight of dimethylformamide |
| Emulsifier: | 2 | parts by weight of alkylaryl polyglycol ether |

A suitable preparation of active compound is prepared by mixing 1 part by weight of active compound with the stated amounts of solvent and emulsifier and diluting the concentrate with emulsifier-containing water to the desired concentration.

Leaves of cabbage (Brassica oleracea) are treated by being immersed in the preparation of active compound at the desired concentration and while still wet are populated with caterpillars of the army worm (Spodoptera frugiperda).

After the desired time, the kill rate in % is determined. 100% means that all of the caterpillars have been killed; 0% means that no caterpillars have been killed.

In this test the following compounds, for example, of the preparation examples show an activity of 500 ppm of ≧80%: I-2-1, I-2-2.

### Example No. 7

Myzus persicae test (MYZUPE)

| | | |
|---|---|---|
| Solvent: | 7 | parts by weight of dimethylformamide |
| Emulsifier: | 2 | parts by weight of alkylaryl polyglycol ether |

A suitable preparation of active compound is prepared by mixing 1 part by weight of active compound with the stated amounts of solvent and emulsifier and diluting the concentrate with emulsifier-containing water to the desired concentration.

Leaves of cabbage (Brassica oleracea) heavily infested by the green peach aphid (Myzus persicae) are treated by being immersed in the preparation of active compound at the desired concentration.

After the desired time, the kill rate in % is determined. 100% means that all of the aphids have been killed; 0% means that no aphids have been killed.

In this test the following compound, for example, of the preparation examples shows an activity of 500 ppm of ≧80%: I-2-1.

### Example No. 8

Tetranychus test, OP-resistent (TETRUR)

| | | |
|---|---|---|
| Solvent: | 7 | parts by weight of dimethylformamide |
| Emulsifier: | 2 | parts by weight of alkylaryl polyglycol ether |

A suitable preparation of active compound is prepared by mixing 1 part by weight of active compound with the stated amounts of solvent and emulsifier and diluting the concentrate with emulsifier-containing water to the desired concentration.

Bean plants (Phaseolus vulgaris) heavily infested by all stages of the greenhouse red spider mite (Tetranychus urticae) are immersed in the preparation of active compound at the desired concentration.

After the desired time, the effect in % is determined. 100% means that all of the spider mites have been killed; 0% means that no spider mites have been killed.

In this test the following compounds, for example, of the preparation examples show an activity of 100 ppm of ≧80%: I-2-1, I-2-2, I-2-3, I-2-4, I-2-10.

### Example No. 9

Meloidogyne test (MELGIN)

| | | |
|---|---|---|
| Solvent: | 7 | parts by weight of dimethylformamide |
| Emulsifier: | 2 | parts by weight of alkylaryl polyglycol ether |

A suitable preparation of active compound is prepared by mixing 1 part by weight of active compound with the stated amounts of solvent and emulsifier and diluting the concentrate with water to the desired concentration.

Containers are filled with sand, a solution of active compound, Meloidogyne incognita egg/larvae suspension and lettuce seeds. The lettuce seeds germinate and the plants develop. On the roots, galls develop.

After the desired time, the nematicidal effect is determined in % from the formation of galls. 100% means that no galls were found; 0% means that the number of galls on the treated plants corresponds to that of the untreated control.

In this test the following compounds, for example, of the preparation examples show an activity of 20 ppm of ≧80%: I-2-1, I-2-2.

### Example No. 10

### 1. Pre-Emergence Herbicidal Effect

Seeds of mono- and dicotyledonous weed and crop plants are placed in sandy loam soil in wood fibre pots and covered with soil. The test compounds, formulated as wettable powders (WP), are then applied as an aqueous suspension with a water application rate of 600 l/ha (converted) and with addition of 0.2% wetting agent, in different dosages to the surface of the covering soil.

After the treatment, the pots are placed in a greenhouse and maintained under optimum growth conditions for the test plants. The visual scoring of the emergence damage on the trial plants is carried out after a trial time of 3 weeks in comparison to untreated controls (herbicidal effect in percent (%): 100% effect=plants have died, 0% effect=like control plants).

### 2. Post-Emergence Herbicidal Effect

Seeds of mono- and dicotyledonous weed and crop plants are placed in sandy loam soil in wood fibre pots, covered with soil and cultivated in a greenhouse under good growth conditions. 2-3 weeks after sowing, the trial plants are treated at the one-leaf stage. The test compounds, formulated as wettable powders (WP), are sprayed at various dosages with a water application rate of 600 l/ha (converted) and with addition of 0.2% wetting agent to the green parts of the plants. After the trial plants have stood in the greenhouse for about 3 weeks under optimum growth conditions, the effect of the products is scored visually in comparison to untreated controls (herbicidal effect in percent (%): 100% effect=plants have died, 0% effect=like control plants).

| Ex. No. | Greenhouse | (g a.i./ha) | Echinochloa | Lolium | Setaria |
|---|---|---|---|---|---|
| I-2-2 | pre-emergence | 1000 | 100 | 80 | 100 |

| Ex. No. | Greenhouse | (g a.i./ha) | Sugarbeets | Digitaria | Echinochloa | Lolium | Setaria | Sorghum |
|---|---|---|---|---|---|---|---|---|
| I-2-2 | post-emergence | 320 | 0 | 100 | 100 | 80 | 100 | 80 |

| Ex. No. | Greenhouse | (g a.i./ha) | Echinochloa | Lolium | Setaria | Sinapis |
|---|---|---|---|---|---|---|
| I-2-6 | post-emergence | 320 | 90 | 70 | 90 | 40 |
| I-2-10 | post-emergence | 320 | 70 | 70 | 90 | 80 |

| Ex. No. | Greenhouse | (g a.i./ha) | Echinochloa | Lolium | Setaria |
|---|---|---|---|---|---|
| I-1-7 | pre-emergence | 320 | — | 100 | 100 |
| I-1-7 | post-emergence | 320 | 90 | 90 | 90 |
| I-1-10 | post-emergence | 320 | 90 | 90 | 80 |

### Example No. 11

### In Vitro Test for ED₅₀ Determination on Microorganisms

A methanolic solution of the active compound under test, admixed with the emulsifier PS 16, is pipetted into the wells of microtitre plates. After the solvent has evaporated, 200 μl of potato dextrose medium are added per well.

The medium has been admixed beforehand with an appropriate concentration of spores or mycelium of the fungus under test.

The resulting concentrations of the active compound are 0.1, 1, 10 and 100 ppm. The resulting concentration of the emulsifier is 300 ppm.

The plates are then incubated on a shaker at a temperature of 20° C. for 3 to 5 days, until sufficient growth can be observed in the untreated control.

Evaluation takes place photometrically at a wavelength of 620 nm. The data for the different concentrations are used to calculate the dose of active compound leading to 50% inhibition of fungal growth as compared with the untreated control (ED₅₀).

TABLE
In vitro test for ED₅₀ determination on microorganisms

| Active compound | Microorganism | ED₅₀ |
|---|---|---|
| Ex. I-2-1 | Pyricularia oryzae | 0.48 |
| Ex. I-2-2 | Pyricularia oryzae | 0.12 |
| Ex. I-1-1 | Pyricularia oryzae | <0.1 |

### Example No. 12

Plasmopara test (grapevine)/protective

| | | |
|---|---|---|
| Solvent: | 24.5 | parts by weight of acetone |
| | 24.5 | parts by weight of dimethylacetamide |
| Emulsifier: | 1 | part by weight of alkylaryl polyglycol ether |

A suitable preparation of active compound is prepared by mixing 1 part by weight of active compound with the stated amounts of solvent and emulsifier and diluting the concentrate with water to the desired concentration.

To test for protective activity, young plants are sprayed with the preparation of active compound at the stated application rate. After the spray coating has dried on, the plants are inoculated with an aqueous spore suspension of Plasmopara viticola and then spend 1 day in an incubation cabin at about 20° C. and 100% relative humidity. Thereafter the plants are placed in a greenhouse for 4 days at about 21° C. and about 90% humidity. The plants are then moistened and placed in an incubation cabin for 1 day.

Evaluation takes place 6 days after inoculation. 0% denotes an efficacy corresponding to that of the control, while an efficacy of 100% denotes a total absence of observed infestation.

TABLE
Plasmopara test (grapevine)/protective

| Active | Application rate of | |
|---|---|---|
| compound | active compound in g/ha | Efficacy in % |
| Example I-2-2 | 100 | 100 |

### Example No. 13

Venturia test (apple)/protective

| | | |
|---|---|---|
| Solvent: | 24.5 | parts by weight of acetone |
| | 24.5 | parts by weight of dimethylacetamide |
| Emulsifier: | 1 | part by weight of alkylaryl polyglycol ether |

A suitable preparation of active compound is prepared by mixing 1 part by weight of active compound with the stated amounts of solvent and emulsifier and diluting the concentrate with water to the desired concentration.

To test for protective activity, young plants are sprayed with the preparation of active compound at the stated application rate. After the spray coating has dried on, the plants are inoculated with an aqueous conidia suspension of the causative organism of apple scab, Venturia inaequalis, and then spend 1 day in an incubation cabin at about 20° C. and 100% relative humidity.

Thereafter the plants are placed in a greenhouse at about 21° C. and about 90% relative humidity.

Evaluation takes place 10 days after inoculation. 0% denotes an efficacy corresponding to that of the control, while an efficacy of 100% denotes a total absence of observed infestation.

TABLE
Venturia test (apple)/protective

| Active | Application rate of | |
|---|---|---|
| compound | active compound in g/ha | Efficacy in % |
| Example I-2-2 | 100 | 99 |

### Example No. 14

Botrytis test (bean)/protective

| | | |
|---|---|---|
| Solvent: | 24.5 | parts by weight of acetone |
| | 24.5 | parts by weight of dimethylacetamide |
| Emulsifier: | 1 | part by weight of alkylaryl polyglycol ether |

A suitable preparation of active compound is prepared by mixing 1 part by weight of active compound with the stated amounts of solvent and emulsifier and diluting the concentrate with water to the desired concentration.

To test for protective activity, young plants are sprayed with the preparation of active compound at the stated application rate. After the spray coating has dried on, two small pieces of agar colonized by Botrytis cinerea are placed on each leaf. The inoculated plants are placed in a darkened chamber at about 20° C. and 100% humidity.

Two days after inoculation, the size of the infestation spots on the leaves is evaluated. 0% denotes an efficacy corresponding to that of the control, while an efficacy of 100% denotes a total absence of observed infestation.

TABLE
Botrytis test (bean)/protective

| Active | Application rate of | |
|---|---|---|
| compound | active compound in g/ha | Efficacy in % |
| Example I-2-2 | 500 | 93 |

### Example No. 15

Critical concentration test/soil insects -
treatment of transgenic plants

| Test insect: | Diabrotica balteata - larvae in soil | |
|---|---|---|
| Solvent: | 24.5 | parts by weight of acetone |
| Emulsifier: | 1 | part by weight of alkylaryl polyglycol ether |

A suitable preparation of active compound is prepared by mixing 1 part by weight of active compound with the stated amount of solvent, adding the stated amount of emulsifier and diluting the concentrate with water to the desired concentration.

The preparation of active compound is poured onto the soil. The concentration of the active compound in the preparation is almost irrelevant here, the only critical factor being the amount by weight of active compound per unit volume of soil, which is reported in ppm (mg/l). The soil is placed in 0.25 l pots which are left to stand at 20° C.

Immediately after sample preparation, 5 pregerminated maize corns of the cultivar YIELD GUARD (trade mark of Monsanto Comp., USA) are placed in each pot. After 2 days the corresponding test insects are placed into the treated soil. After a further 7 days the efficacy of the active compound is determined by counting the maize plants that have emerged (1 plant=20% effect).

### Example No. 16

Heliothis virescens test - treatment of transgenic plants

| | | |
|---|---|---|
| Solvent: | 7 | parts by weight of acetone |
| Emulsifier: | 1 | part by weight of alkylaryl polyglycol ether |

An appropriate preparation of active compound is prepared by mixing 1 part by weight of active compound with the stated amount of solvent and the stated amount of emulsifier and diluting the concentrate with water to the desired concentration.

Soya bean shoots (Glycine max) of the cultivar Roundup Ready (trade mark of Monsanto Comp. USA) are treated by being immersed in the preparation of active compound at the desired concentration and are populated with the tobacco bugworm Heliothis virescens while the leaves are still moist.

After the desired time, the kill of the insects is determined.

1. A compound of formula (I) 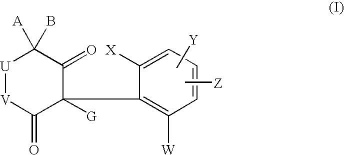
in which
U represents oxygen or represents a group 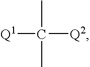
V represents oxygen or represents a group 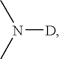
with the proviso that one but only one of U or V must represent oxygen,
W represents hydrogen, halogen, alkyl, alkenyl, alkynyl, alkoxy, alkenyloxy, haloalkyl or haloalkoxy,
X represents halogen, alkyl, alkenyl, alkynyl, alkoxy, alkenyloxy, alkylthio, alkylsulphinyl, alkylsulphonyl, haloalkyl, haloalkoxy, haloalkenyloxy, nitro or cyano,
Y represents hydrogen, halogen, alkyl, alkoxy, alkenyloxy, haloalkyl, haloalkoxy, haloalkenyloxy, nitro or cyano,
Z represents hydrogen, alkyl, alkenyl, alkynyl, alkoxy, halogen or in each case optionally substituted aryl or heteroaryl,
A represents hydrogen or in each case optionally halogen-substituted alkyl, alkenyl, alkoxyalkyl, polyalkoxyalkyl, alkylthioalkyl, or saturated or unsaturated optionally substituted cycloalkyl, in which optionally at least one ring atom is replaced by a heteroatom,
B represents hydrogen, alkyl or alkoxyalkyl, or
A and B, together with the carbon atom to which they are attached, represent a saturated or unsaturated, unsubstituted or substituted ring optionally containing at least one heteroatom,
D represents hydrogen or an optionally substituted radical selected from the group consisting of alkyl, alkenyl, alkoxyalkyl, polyalkoxyalkyl, alkylthioalkyl and saturated or unsaturated cycloalkyl in which optionally one or more ring atom is replaced by a heteroatom,
G represents halogen or nitro,
A and Q¹ together represent optionally substituted alkanediyl in which two carbon atoms that are not directly adjacent optionally form a further optionally substituted ring which may optionally be interrupted by a heteroatom,
Q¹ represents hydrogen, alkyl, alkoxy, alkoxyalkyl, optionally substituted cycloalkyl in which optionally a methylene group has been replaced by oxygen or sulphur, or in each case optionally substituted phenyl, heteroaryl, phenylalkyl or heteroarylalkyl,
Q² represents hydrogen or alkyl,
Q¹ and Q², together with the carbon atom to which they are attached, represent a saturated or unsaturated, unsubstituted or substituted ring optionally containing a heteroatom.

2. A compound of formula (I) according to claim 1, in which
W represents hydrogen, C₁-C₆-alkyl, C₂-C₆-alkenyl, ethynyl, fluorine, chlorine, bromine, C₁-C₄-haloalkyl, C₁-C₆-alkoxy or C₁-C₄-haloalkoxy,
X represents fluorine, chlorine, bromine, C₁-C₆-alkyl, C₁-C₄-haloalkyl, C₁-C₆-alkoxy, C₂-C₆-alkenyl, ethynyl, C₁-C₄-haloalkoxy, nitro or cyano,
Y represents hydrogen, fluorine, chlorine, bromine, C₁-C₆-alkyl, C₁-C₄-haloalkyl, C₁-C₆-alkoxy, C₁-C₄-haloalkoxy or cyano,
Z represents hydrogen, C₁-C₆-alkyl, fluorine, chlorine, bromine, C₁-C₆-alkoxy, C₂-C₆-alkenyl, ethynyl or represents one of the radicals 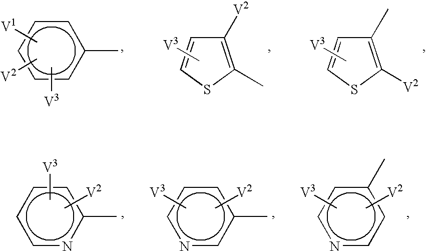
V¹ represents hydrogen, halogen, C₁-C₂-alkyl, C₁-C₆-alkoxy, C₁-C₆-alkylthio, C₁-C₄-haloalkyl, C₁-C₄-haloalkoxy, nitro or cyano,
V² represents hydrogen, fluorine, chlorine, C₁-C₆-alkyl, C₁-C₆-alkoxy, C₁-C₄-haloalkyl or C₁-C₄-haloalkoxy,
V³ represents hydrogen, fluorine, chlorine, methyl or methoxy,
with the proviso that W, X and Y do not represent bromine, C₂-C₆-alkenyl or ethynyl if Z represents V¹-, V²- and V³-substituted phenyl or heteroaryl, and
with the proviso that only a maximum of two of the radicals W, X and Z is C₂-C₆-alkenyl or ethynyl, and in that case none of W, X, Y and Z is bromine,
A represents hydrogen or in each case optionally halogen-substituted C₁-C₁₂-alkyl, C₃-C₈-alkenyl, C₁-C₁₀-alkoxy-C₁-C₈-alkyl, poly-C₁-C₈-alkoxy-C₁-C₈-alkyl or C₁-C₁₀-alkylthio-C₁-C₆-alkyl, optionally halogen-, C₁-C₆-alkyl- or C₁-C₆-alkoxy-substituted C₃-C₈-cycloalkyl, in which optionally one or two ring atoms that are not directly adjacent are replaced by oxygen or sulphur,
B represents hydrogen, C₁-C₁₂-alkyl, or C₁-C₈-alkoxy-C₁-C₆-alkyl, or
A, B and the carbon atom to which they are attached represent saturated C₃-C₁₀-cycloalkyl or unsaturated C₅-C₁₀-cycloalkyl, in which optionally a ring atom is replaced by oxygen or sulphur and which are optionally substituted once or twice by C₁-C₈-alkyl, C₃-C₁₀-cycloalkyl, C₁-C₈-haloalkyl, C₁-C₈-alkoxy, C₁-C₈-alkylthio, halogen or phenyl,
D represents hydrogen or in each case optionally halogen-substituted C₁-C₂-alkyl, C₃-C₈-alkenyl, C₁-C₁₀-alkoxy-C₂-C₈-alkyl, poly-C₁-C₈-alkoxy-C₂-C₈-alkyl or C₁-C₁₀-alkylthio-C₂-C₈-alkyl, or optionally halogen-, C₁-C₄-alkyl-, C₁-C₄-alkoxy- or C₁-C₄-haloalkyl-substituted C₄-C₈-cycloalkyl, in which optionally a ring atom is replaced by oxygen or sulphur,
G represents halogen or nitro,
A and Q¹ together represent C₃-C₆-alkanediyl which is optionally substituted once or twice by C₁-C₄-alkyl or C₁-C₄-alkoxy and which may optionally be interrupted by oxygen or sulphur,
Q¹ represents hydrogen, C₁-C₆-alkyl, C₁-C₆-alkoxy, C₁-C₆-alkoxy-C₁-C₂-alkyl or C₁-C₆-alkylacyloxy, or optionally fluorine-, chlorine-, C₁-C₄-alkyl-, C₁-C₂-haloalkyl- or C₁-C₄-alkoxy-substituted C₃-C₈-cycloalkyl, in which optionally a methylene group has been replaced by oxygen or sulphur, or phenyl optionally substituted by halogen, C₁-C₄-alkyl, C₁-C₄-alkoxy, C₁-C₂-haloalkyl, C₁-C₂-haloalkoxy, cyano or nitro,
Q² represents hydrogen or C₁-C₄-alkyl, or
Q¹ and Q², together with the carbon atom to which they are attached, represent optionally C₁-C₆-alkyl-, C₁-C₆-alkoxy- or C₁-C₂-haloalkyl-substituted C₃-C₇-cycloalkyl, in which optionally a ring atom is replaced by oxygen or sulphur.

3. A compound of formula (I) according to claim 1, in which
W represents hydrogen, C₁-C₄-alkyl, C₂-C₃-alkenyl, ethynyl, fluorine, chlorine, bromine, trifluoromethyl or C₁-C₄-alkoxy,
X represents fluorine, chlorine, bromine, C₁-C₄-alkyl, C₁-C₄-alkoxy, C₂-C₃-alkenyl, ethynyl, C₁-C₂-haloalkyl, C₁-C₂-haloalkoxy or cyano,
Y represents hydrogen, fluorine, chlorine, bromine, C₁-C₄-alkyl, C₁-C₂-haloalkyl, C₁-C₄-alkoxy or C₁-C₂-haloalkoxy,
Z represents hydrogen, C₁-C₄-alkyl, fluorine, chlorine, bromine, C₁-C₄-alkoxy, C₂-C₃-alkenyl, ethynyl, or represents the radical 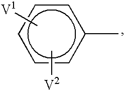
V¹ represents hydrogen, fluorine, chlorine, bromine, C₁-C₆-alkyl, C₁-C₄-alkoxy, C₁-C₄-alkylthio, C₁-C₂-haloalkyl or C₁-C₂-haloalkoxy,
V² represents, hydrogen, fluorine, chlorine, C₁-C₄-alkyl, C₁-C₄-alkoxy, C₁-C₂-haloalkyl or C₁-C₂-haloalkoxy,
with the proviso that W, X and Y do not represent bromine, C₂-C₃-alkenyl or ethynyl if Z represents V¹- and V²-substituted phenyl, and
with the proviso that only a maximum of two of the radicals W, X and Z is C₂-C₆-alkenyl or ethynyl, and in that case none of W, X, Y and Z is bromine,
A represents hydrogen, C₁-C₁₀-alkyl or C₁-C₈-alkoxy-C₁-C₆-alkyl, the latter two of which are each optionally substituted one to three times by fluorine, or chlorine, or represents C₃-C₇-cycloalkyl optionally substituted once or twice by fluorine, chlorine, C₁-C₄-alkyl or C₁-C₄-alkoxy, and in which optionally a ring atom is replaced by oxygen or sulphur,
B represents hydrogen or C₁-C₆-alkyl or
A, B and the carbon atom to which they are attached represent saturated or unsaturated C₃-C₇-cycloalkyl, in which optionally a ring atom is replaced by oxygen or sulphur, and which is optionally monosubstituted by C₁-C₆-alkyl, C₁-C₃-haloalkyl or C₁-C₆-alkoxy,
D represents hydrogen, represents C₁-C₁₀-alkyl, C₃-C₆-alkenyl, C₁-C₄-alkoxy-C₂-C₄-alkyl or C₁-C₄-alkylthio-C₂-C₄-alkyl, each of which is optionally substituted one to three times by fluorine or chlorine, or represents C₄-C₇-cycloalkyl which is optionally substituted by fluorine, chlorine, C₁-C₄-alkyl, C₁-C₄-alkoxy or C₁-C₄-haloalkyl, and in which optionally a methylene group is replaced by oxygen or sulphur,
G represents bromine, chlorine or nitro,
A and Q¹ together represent C₃-C₄-alkanediyl optionally monosubstituted by C₁-C₂-alkyl,
Q¹ represents hydrogen, C₁-C₄-alkyl, C₁-C₄-alkoxy or C₁-C₄-alkoxy-C₁-C₂-alkyl, or C₃-C₆-cycloalkyl which is optionally monosubstituted by methyl or methoxy, and in which optionally a methylene group is replaced by oxygen,
Q² represents hydrogen, methyl or ethyl or
Q¹ and Q², together with the carbon to which they are attached, represent saturated C₅-C₆-cycloalkyl which is optionally monosubstituted by C₁-C₄-alkyl or C₁-C₄-alkoxy, and in which optionally a ring atom is replaced by oxygen.

4. A compound of the formula (I) according to claim 1, in which
W represents hydrogen, fluorine, chlorine, bromine, methyl, ethyl, n-propyl or methoxy,
X represents fluorine, chlorine, bromine, methyl, ethyl, n-propyl, isopropyl, methoxy, ethoxy, n-propoxy, trifluoromethyl, difluoromethoxy, trifluoromethoxy or cyano,
Y represents hydrogen, fluorine, chlorine, bromine, methyl, ethyl, methoxy or trifluoromethyl,
Z represents hydrogen, methyl, ethyl, n-propyl, isopropyl, fluorine, chlorine, bromine, methoxy or represents the radical 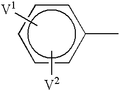
V¹ represents hydrogen, fluorine, chlorine, bromine, methyl, ethyl, n-propyl, isopropyl, n-butyl, isobutyl, tert-butyl, methoxy, ethoxy, n-propoxy, isopropoxy, trifluoromethyl or trifluoromethoxy,
V² represents hydrogen, fluorine, chlorine, methyl, methoxy or trifluoromethyl,
A represents hydrogen, C₁-C₄-alkyl or C₁-C₄-alkoxy-C₁-C₂-alkyl, or C₃-C₆-cycloalkyl which is optionally monosubstituted by fluorine, methyl, ethyl or methoxy, and in which optionally a ring atom is replaced by oxygen or sulphur,
B represents hydrogen, methyl or ethyl, or
A, B and the carbon atom to which they are attached represent saturated C₃-C₆-cycloalkyl in which optionally a ring atom is replaced by oxygen and which is optionally monosubstituted by methyl, ethyl, trifluoromethyl, methoxy, ethoxy, n-propoxy, n-butoxy or isobutoxy,
D represents hydrogen or represents C₁-C₈-alkyl, C₃-C₄-alkenyl, C₁-C₆-alkoxy-C₂-C₄-alkyl, C₁-C₄-alkylthio-C₂-C₄-alkyl or C₄-C₆-cycloalkyl, each of which is optionally substituted one to three times by fluorine,
G represents chlorine or nitro,
A and Q¹ together represent C₃-C₄-alkanediyl optionally monosubstituted by methyl,
Q¹ represents hydrogen, methyl, ethyl, n-propyl, isopropyl, methoxy, ethoxy, n-propoxy, cyclopropyl, cyclopentyl or cyclohexyl,
Q² represents hydrogen, methyl or ethyl, or
Q¹ and Q², together with the carbon to which they are attached, represent saturated C₅-C₆-cycloalkyl which is optionally substituted by methyl, ethyl, n-propyl, isopropyl, methoxy, ethoxy, propoxy or butoxy, and in which optionally a ring atom is replaced by oxygen.

5. A compound of formula (I) according to claim 1, in which
W represents hydrogen, chlorine, methyl or ethyl,
X represents chlorine, bromine, methyl or ethyl,
Y represents hydrogen, chlorine, bromine or methyl,
Z represents methyl or represents the radical 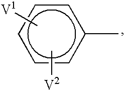
V¹ represents hydrogen, fluorine, chlorine or trifluoromethyl,
V² represents hydrogen, fluorine, chlorine or trifluoromethyl,
A represents C₁-C₄-alkyl,
B represents methyl,
A, B and the carbon atom to which they are attached represent saturated C₃-C₆-cycloalkyl in which optionally a ring atom is replaced by oxygen,
D represents methyl,
G represents chlorine,
A and Q¹ together represent C₃-C₄-alkanediyl,
Q¹ represents hydrogen or methyl,
Q² represents hydrogen or methyl.

6. A process for preparing a compound of formula (I) according to claim 1, comprising
A) obtaining a compound of formula (I-1) or (I-2) 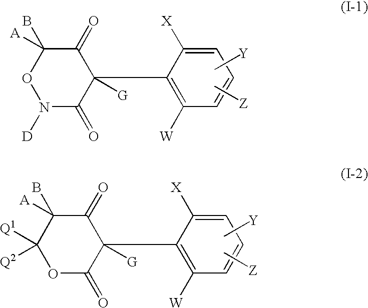
in which
A, B, D, Q¹, Q², W, X, Y and Z have the definitions indicated in claim 1
and
G represents halogen
by reacting a compound of formula (II-1) or (II-2) 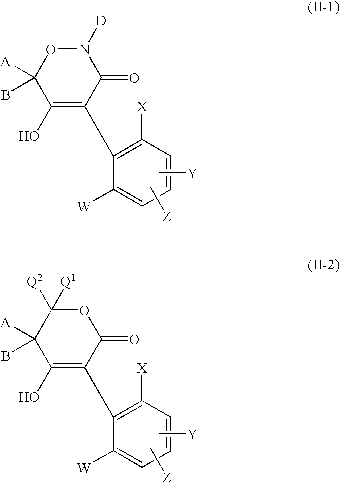
in which
A, B, D, Q¹, Q², W, X, Y and Z have the definition indicated in claim 1,
with a halogenating agent in the presence of a solvent and optionally in the presence of a free-radical initiator; or
B) obtaining a compound of formula (I-1) or (I-2) 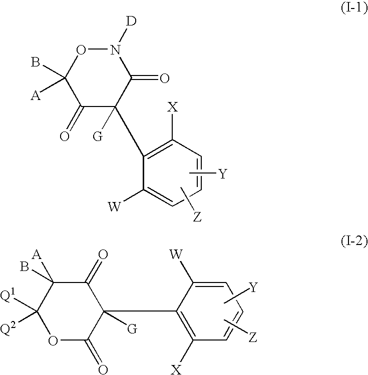
in which
A, B, D, Q¹, Q², W, X, Y and Z have the definitions indicated in claim 1
and
G represents nitro
by reacting a compound of formula (II-1) or (II-2) 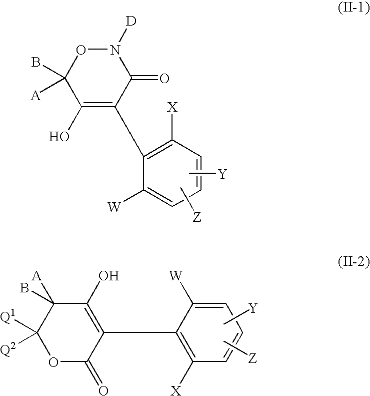
in which
A, B, D, Q¹, Q², W, X, Y and Z have the definition indicated in claim 1, with a nitrating agent in the presence of a solvent.

7. A pesticide, herbicide or microbicide composition comprising at least one compound of formula (I) according to claim 1.

8. A method of controlling animal pests, unwanted vegetation or microbes comprising contacting pests, unwanted vegetation, microbes or their habitat with a compound of formula (I) according to claim 1.

9. (canceled)

10. A process for producing a pesticide, herbicide or microbicide composition comprising mixing a compound of formula (I) according to claim 1 with an extender or a surface active substance, or a mixture thereof.

11. (canceled)

12. A composition comprising an effective amount of an active-compound combination comprising
(a′) at least one phenyl-substituted [1,2]-oxazinedione derivative of formula (I-1) or one phenyl-substituted dihydropyrone derivative of formula (I-2), 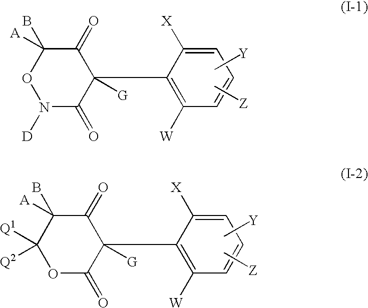
in which A, B, D, G, Q¹, Q², W, X, Y and Z are as defined in claim 1
and
(b′) at least one crop plant tolerance promoter compound selected from the group consisting of:
4-dichloroacetyl-1-oxa-4-azaspiro[4.5]decane (AD-67, MON-4660), 1-dichloro-acetylhexahydro-3,3,8a-trimethylpyrrolo[1,2-a]pyrimidin-6(2H)-one (dicyclonon, BAS-145138), 4-dichloroacetyl-3,4-dihydro-3-methyl-2H-1,4-benzoxazine (benoxacor), 1-methylhexyl 5-chloroquinoline-8-oxyacetate (cloquintocet-mexyl), 3-(2-chlorobenzyl)-1-(1-methyl-1-phenylethyl)urea (cumyluron), α-(cyanomethoximino)phenylacetonitrile (cyometrinil), 2,4-dichlorophenoxyacetic acid (2,4-D), 4-(2,4-dichlorophenoxy)butyric acid (2,4-DB), 1-(1-methyl-1-phenylethyl)-3-(4-methylphenyl)urea (daimuron, dymron), 3,6-dichloro-2-methoxybenzoic acid (dicamba), S-1-methyl-1-phenylethyl piperidine-1-thiocarboxylate (dimepiperate), 2,2-dichloro-N-(2-oxo-2-(2-propenylamino)ethyl)-N-(2-propenyl)acetamide (DKA-24), 2,2-dichloro-N,N-di-2-propenylacetamide (dichlormid), 4,6-dichloro-2-phenylpyrimidine (fenclorim), ethyl 1-(2,4-dichlorophenyl)-5-trichloromethyl-1H-1,2,4-triazole-3-carboxylate (fenchlorazole-ethyl), phenylmethyl 2-chloro-4-trifluoromethylthiazole-5-carboxylate (flurazole), 4-chloro-N-(1,3-dioxolan-2-ylmethoxy)-α-trifluoroacetophenone oxime (fluxofenim), 3-dichloroacetyl-5-(2-furanyl)-2,2-dimethyloxazolidine (furilazole, MON-13900), ethyl 4,5-dihydro-5,5-diphenyl-3-isoxazolecarboxylate (isoxadifen-ethyl), 1-(ethoxycarbonyl)ethyl 3,6-dichloro-2-methoxybenzoate (lactidichlor), (4-chloro-o-tolyloxy)acetic acid (MCPA), 2-(4-chloro-o-tolyloxy)propionic acid (mecoprop), diethyl 1-(2,4-dichorophenyl)-4,5-dihydro-5-methyl-1H-pyrazole-3,5-dicarboxylate (mefenpyr-diethyl), 2-dichloromethyl-2-methyl-1,3-dioxolane (MG-191), 2-propenyl-1-oxa-4-azaspiro[4.5]decane-4-carbodithioate (MG-838), 1,8-naphthalic anhydride, α-(1,3-dioxolan-2-ylmethoximino)phenylacetonitrile (oxabetrinil), 2,2-dichloro-N-(1,3-dioxolan-2-ylmethyl)-N-(2-propenyl)acetamide (PPG-1292), 3-dichloroacetyl-2,2-di-methyloxazolidine (R-28725), 3-dichloroacetyl-2,2,5-trimethyloxazolidine (R-29148), 4-(4-chloro-o-tolyl)butyric acid, 4-(4-chlorophenoxy)butyric acid, diphenylmethoxyacetic acid, methyl diphenylmethoxyacetate, ethyl diphenylmethoxyacetate, methyl 1-(2-chlorophenyl)-5-phenyl-1H-pyrazole-3-carboxylate, ethyl 1-(2,4-dichlorophenyl)-5-methyl-1H-pyrazole-3-carboxylate, ethyl 1-(2,4-dichlorophenyl)-5-isopropyl-1H-pyrazole-3-carboxylate, ethyl 1-(2,4-dichlorophenyl)-5-(1,1-dimethylethyl)-1H-pyrazole-3-carboxylate, ethyl 1-(2,4-dichlorophenyl)-5-phenyl-1H-pyrazole-3-carboxylate, ethyl 5-(2,4-dichlorobenzyl)-2-isoxazoline-3-carboxylate, ethyl 5-phenyl-2-isoxazoline-3-carboxylate, ethyl 5-(4-fluorophenyl)-5-phenyl-2-isoxazoline-3-carboxylate, 1,3-dimethylbut-1-yl 5-chloroquinoline-8-oxyacetate, 4-allyloxybutyl 5-chloroquinoline-8-oxyacetate, 1-allyloxyprop-2-yl 5-chloroquinoline-8-oxyacetate, methyl 5-chloroquinoxaline-8-oxyacetate, ethyl 5-chloroquinoline-8-oxyacetate, allyl 5-chloroquinoxaline-8-oxyacetate, 2-oxoprop-1-yl 5-chloroquinoline-8-oxyacetate, diethyl 5-chloroquinoline-8-oxymalonate, diallyl 5-chloroquinoxaline-8-oxymalonate, diethyl 5-chloroquinoline-8-oxymalonate, 4-carboxychroman-4-ylacetic acid (AC-304415), 4-chlorophenoxyacetic acid, 3,3′-dimethyl-4-methoxybenzophenone, 1-bromo-4-chloromethylsulphonylbenzene, 1-[4-(N-2-methoxybenzoylsulphamoyl)phenyl]-3-methylurea (N-(2-methoxybenzoyl)-4-[(methylaminocarbonyl)-amino]benzenesulphonamide), 1-[4-(N-2-methoxybenzoylsulphamoyl)phenyl]-3,3-dimethylurea, 1-[4-(N-4,5-dimethylbenzoylsulphamoyl)phenyl]-3-methylurea, 1-[4-(N-naphthylsulphamoyl)phenyl]-3,3-dimethylurea, N-(2-methoxy-5-methylbenzoyl)-4-(cyclopropylaminocarbonyl)benzenesulphonamide,
a compound
of the general formula (IIa) 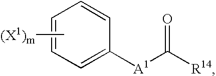
of the general formula (IIb) 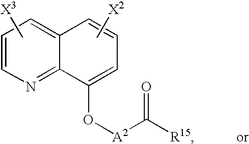
of the general formula (IIe) 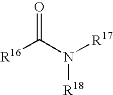
where
m represents a number 0, 1, 2, 3, 4 or 5,
A¹ represents one of the divalent heterocyclic groupings shown below 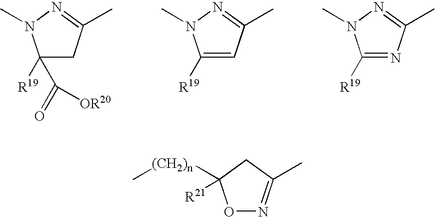
n represents a number between 1, 2, 3, 4 or 4,
A² represents optionally C₁-C₄-alkyl-, C₁-C₄-alkoxy-carbonyl- or C₁-C₄-alkenyloxy-carbonyl-substituted alkanediyl having 1 or 2 carbon atoms,
R¹⁴ represents hydroxyl, mercapto, amino, C₁-C₆-alkoxy, C₁-C₆-alkylthio, C₁-C₆-alkylamino or di-(C₁-C₄-alkyl)-amino,
R¹⁵ represents hydroxyl, mercapto, amino, C₁-C₇-alkoxy, C₁-C₆-alkenyloxy, C₁-C₆-alkenyloxy-C₁-C₆-alkoxy, C₁-C₆-alkylthio, C₁-C₆-alkylamino or di-(C₁-C₄-alkyl)-amino,
R¹⁶ represents optionally fluorine-, chlorine- or bromine-substituted C₁-C₄-alkyl,
R¹⁷ represents hydrogen, in each case optionally fluorine-, chlorine- or bromine-substituted C₁-C₆-alkyl, C₂-C₆-alkenyl or C₂-C₆-alkynyl, C₁-C₄-alkoxy-C₁-C₄-alkyl, dioxolanyl-C₁-C₄-alkyl, furyl, furyl-C₁-C₄-alkyl, thienyl, thiazolyl, piperidinyl, or optionally fluorine-, chlorine-, bromine- or C₁-C₄-alkyl-substituted phenyl,
R¹⁸ represents hydrogen, in each case optionally fluorine-, chlorine- or bromine-substituted C₁-C₆-alkyl, C₂-C₆-alkenyl or C₂-C₆-alkynyl, C₁-C₄-alkoxy-C₁-C₄-alkyl, dioxolanyl-C₁-C₄-alkyl, furyl, furyl-C₁-C₄-alkyl, thienyl, thiazolyl, piperidinyl, or optionally fluorine-, chlorine- or bromine- or C₁-C₄-alkyl-substituted phenyl,
R¹⁷ and R¹⁸ also together represent C₃-C₆-alkanediyl or C₂-C₅-oxaalkanediyl, each of which is optionally substituted by C₁-C₄-alkyl, phenyl, furyl, a fused benzene ring or by two substituents which, together with the C atom to which they are attached, form a 5- or 6-membered carbocycle,
R¹⁹ represents hydrogen, cyano, or halogen, or represents in each case optionally fluorine-, chlorine- or bromine-substituted C₁-C₄-alkyl, C₃-C₆-cycloalkyl or phenyl,
R²⁰ represents hydrogen, optionally hydroxyl-, cyano-, halogen- or C₁-C₄-alkoxy-substituted C₁-C₆-alkyl, C₃-C₆-cycloalkyl or tri-(C₁-C₄-alkyl)-silyl,
R²¹ represents hydrogen, cyano, halogen, or represents in each case optionally fluorine-, chlorine- or bromine-substituted C₁-C₄-alkyl, C₃-C₆-cycloalkyl or phenyl,
X¹ represents nitro, cyano, halogen, C₁-C₄-alkyl, C₁-C₄-haloalkyl, C₁-C₄-alkoxy or C₁-C₄-haloalkoxy,
X² represents hydrogen, cyano, nitro, halogen, C₁-C₄-alkyl, C₁-C₄-haloalkyl, C₁-C₄-alkoxy or C₁-C₄-haloalkoxy,
X³ represents hydrogen, cyano, nitro, halogen, C₁-C₄-alkyl, C₁-C₄-haloalkyl, C₁-C₄-alkoxy or C₁-C₄-haloalkoxy,
and a compound
of the general formula (IId) 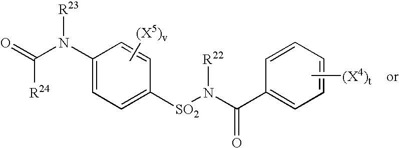
of the general formula (IIe) 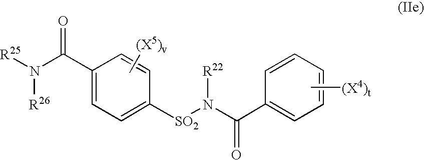
where
t represents a number between 0 and 5,
v represents a number between 0 and 5,
R²² represents hydrogen or C₁-C₄-alkyl,
R²³ represents hydrogen or C₁-C₄-alkyl,
R²⁴ represents hydrogen, in each case optionally cyano-, halogen- or C 1-C₄-alkoxy-substituted C₁-C₆-alkyl, C₁-C₆-alkoxy, C 1-C₆-alkylthio, C₁-C₆-alkylamino or di-(C₁-C₄-alkyl)-amino, or in each case optionally cyano-, halogen- or C₁-C₄-alkyl-substituted C₃-C₆-cycloalkyl, C₃-C₆-cycloalkyloxy, C₃-C₆-cycloalkylthio or C₃-C₆-cycloalkylamino,
R²⁵ represents hydrogen, optionally cyano-, hydroxyl-, halogen- or C₁-C₄-alkoxy-substituted C₁-C₆-alkyl, in each case optionally cyano- or halogen-substituted C₃-C₆-alkenyl or C₃-C₆-alkynyl, or optionally cyano-, halogen- or C₁-C₄-alkyl-substituted C₃-C₆-cycloalkyl,
R²⁶ represents hydrogen, optionally cyano-, hydroxyl-, halogen- or C₁-C₄-alkoxy-substituted C₁-C₆-alkyl, in each case optionally cyano- or halogen-substituted C₃-C₆-alkenyl or C₃-C₆-alkynyl, optionally cyano-, halogen- or C₁-C₄-alkyl-substituted C₃-C₆-cycloalkyl, optionally nitro-, cyano-, halogen-, C₁-C₄-alkyl-, C₁-C₄-haloalkyl-, C₁-C₄-alkoxy- or C₁-C₄-haloalkoxy-substituted phenyl, or together with R²⁵ represents in each case optionally C₁-C₄-alkyl-substituted C₂-C₆-alkanediyl or C₂-C₅-oxaalkanediyl,
X⁴ represents nitro, cyano, carboxyl, carbamoyl, formyl, sulphamoyl, hydroxyl, amino, halogen, C₁-C₄-alkyl, C₁-C₄-haloalkyl, C₁-C₄-alkoxy or C₁-C₄-haloalkoxy, and
X⁵ represents nitro, cyano, carboxyl, carbamoyl, formyl, sulphamoyl, hydroxyl, amino, halogen, C₁-C₄-alkyl, C₁-C₄-haloalkyl, C₁-C₄-alkoxy or C₁-C₄-haloalkoxy.

13. A composition according to claim 12, in which the crop plant tolerance promoter compound is selected from the group consisting of:
cloquintocet-mexyl, fenchlorazole-ethyl, isoxadifen-ethyl, mefenpyr-diethyl, furilazole, fenclorim, cumyluron, dymron,

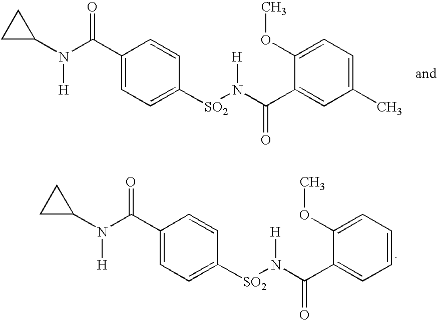

14. A composition according to claim 13, in which the crop plant tolerance promoter compound is cloquintocet-mexyl or mefenpyr-diethyl.

15. A method of controlling unwanted plant growth, comprising contacting a plant or its habitat with a composition according to claim 12.

16. (canceled)

17. A method of controlling unwanted plant growth, comprising contacting a plant or its surroundings with a compound of formula (I) according to claim 1 and a crop plant tolerance promoter compound separately in close temporal succession or in a mixture.
